(19) United States
(12) Patent Application Publication
(10) Pub. No.: US 2012/0093855 A1
(43) Pub. Date: Apr. 19, 2012

(54) RSV F VLPs AND METHODS OF MANUFACTURE AND USE THEREOF

(75) Inventor: Joel R. Haynes, Bozeman, MT (US)

(73) Assignee: LIGOCYTE PHARMACEUTICALS, INC., Bozeman, MT (US)

(21) Appl. No.: 13/140,817

(22) PCT Filed: Nov. 18, 2009
(86) PCT No.: PCT/US2009/064941

§ 371 Date: Nov. 18, 2011

Related U.S. Application Data
(60) Provisional application No. 61/115,780, filed on Nov. 18, 2008.

(51) Int. Cl. A61K 39/12; A61P 31/12; A61P 37/02; C12P 21/06; C07K 14/005

(52) U.S. Cl. 424/204.1; 435/69.1; 530/350; 530/395

(57) ABSTRACT

The present invention relates to the field of isolation of enveloped virus-based virus-like particles (VLPs) free of infectious agents. In preferred examples, the field includes methods of inactivation of infectious agents that do not adversely affect the immunogenicity of the enveloped virus-based VLPs. In certain embodiments, the enveloped virus-based VLPs are produced in insect cell based expression systems.

### FIELD OF THE INVENTION

The present invention relates to the field of isolation of enveloped virus-based virus-like particles (VLPs) free of infectious agents. In preferred examples, the field includes methods of inactivation of infectious agents that do not adversely affect the immunogenicity of the enveloped virus-based VLPs. In certain embodiments, the enveloped virus-based VLPs are produced in insect cell based expression systems.

### BACKGROUND OF THE INVENTION

Respiratory syncytial virus (RSV) is a leading cause of bronchiolitis and pneumonia among infants and children under 1 year of age (CDC National Center for Infectious Diseases (2004) Respiratory Syncytial Virus). RSV can also be a significant lower respiratory tract pathogen in imuno-compromised adults and the elderly. Individuals can be infected multiple times as natural infection with RSV does not induce protective immunity.

RSV is a negative-sense, single-stranded RNA virus that belongs to the genus Pneumovirus of the family Paramyxoviridae. The RSV genome is surrounded by a helical nucleocapsid and encodes at least ten proteins: three transmembrane structural proteins (F, G, and SH), two matrix proteins (M and M2), three nucleocapsid proteins (N, P, and L), and two nonstructural proteins (NS1 and NS2) (Collins et al (1996) Respiratory syncytial virus, pp. 1313-1351, In B. N. Fields (ed.), Fields virology. Raven Press, New York, N.Y.). Neutralizing antibodies appear to be elicited only by the F and G proteins. RSV is divided into subgroups A and B based on the G protein, whereas F is more closely related between the subgroups. Monoclonal antibodies against the F protein have been shown to have neutralizing effect in vitro and prophylactic effects in vivo (e.g. Anderson et al. 1988. J. Virol. 62:4232-4238; Anderson et al. 1986. J. Clin. Micro. 23:475-480; Beeler and Coelingh 1989. J. Virol. 63:2941-50; Garcia-Barreno et al. 1989. J. Virol. 63:925-32; Taylor et al. 1984. Immunology 52: 137-142; and U.S. Pat. No. 6,818,216).

No safe and effective vaccine exists for RSV despite several decades of research. A formalin-inactivated virus vaccine tested in infants and children did not protect against infection and was associated with increased risk of severe symptoms during subsequent infections by wild-type RSV virus (Kapikian et al., 1969, Am. J. Epidemiol. 89:405-21; Chin et al., 1969, Am. J. Epidemiol. 89:449-63). Later attempts focused on developing live-attenuated temperature sensitive mutants also failed due to the inability to identify virus candidates at an appropriate level of attenuation and to the genetic instability of some candidates (Hodes et al. (1974) Proc. Soc. Exp. Biol. Med., 145, 1158-1164; Kim et al. (1973) Pediatrics, 52, 56-63; Wright et al (1976) J. Pediatrics, 88, 931-936).

Virus-like particles (VLPs) offer several advantages over conventional vaccine technology. An important advantage of VLPs for vaccine development is that they mimic native viruses in terms of three-dimensional structure and the ability to induce neutralizing antibody responses to both primary and conformational epitopes and therefore should prove more immunogenic than other vaccine formulations. Unlike viral vectored approaches, VLPs exhibit no problem with pre-existing immunity, thus allowing for recurrent use. VLPs containing RSV antigens have been generated by co-expression of RSV F protein with RSV M protein or with a heterologous M protein in insect cells (US 2008/0233150). However, US 2008/0233150 does not teach actual expression of the RSV F protein by itself in a mammalian cell system where the RSV F protein alone is capable of generating VLPs that are not aggregated with themselves or associated with the viral vector used to express the RSV F protein. Thus, there is a need for a method of generating VLPs that express RSV F protein alone.

### SUMMARY

Preferred embodiments of the present invention meet this need by providing various methods and compositions as disclosed herein for methods of generating respiratory syncytial virus F polypeptide VLPs which do not require additional enveloped virus core forming polypeptides and compositions comprising respiratory syncytial virus F polypeptide VLP preparations. In preferred embodiments, the respiratory syncytial virus F polypeptide VLPs will have one or more of the following additional characteristics: the VLPs will not comprise an enveloped virus core; the VLPs are pleomorphic or of non-uniform size or shape, the VLPs will not comprise an enveloped virus core forming polypeptide. Such preferred embodiments are based upon the surprising observation that the respiratory syncytial virus F polypeptide by itself is able to form VLPs. VLPs formed by the respiratory syncytial virus F polypeptide by itself do not have the protein core typical of enveloped viruses and VLPs formed using enveloped virus components.

An aspect of the invention includes preparations of respiratory syncytial virus F polypeptide virus-like particles comprising a respiratory syncytial virus F polypeptide, wherein the virus-like particles includes one or more of the following characteristics: the virus-like particles not comprise an enveloped virus core; the virus-like particles are pleomorphic, of non-uniform size, or of non-uniform shape; the virus-like particles do not comprise an enveloped virus core forming polypeptide; and/or the virus-like particles comprise mammalian glycosylation. In certain embodiments, virus-like particles may be substantially non-aggregated with the other virus-like particles. In another embodiment that may be combined with the preceding embodiment or aspects, the virus-like particles are substantially not associated with viral vector particles.

In another embodiment that may be combined with any of the preceding embodiments or aspects, the preparation also includes an adjuvant in admixture with the virus-like particles. In another embodiment that may be combined with any of the preceding embodiments or aspects that include an adjuvant, the adjuvant may be located outside the virus-like particle or may be located inside the virus-like particle. In another embodiment that may be combined with any of the preceding embodiments or aspects that include an adjuvant, the adjuvant may be covalently linked to the respiratory syncytial virus F polypeptide to form a covalent linkage.

In another embodiment that may be combined with any of the preceding embodiments or aspects, a neutralizing anti-RSV-F antibody may bind to the respiratory syncytial virus F polypeptide (demonstrating that the RSV F polypeptide is in substantially a native conformation). In certain embodiments that may be combined with any of the preceding embodiments or aspects that include such a neutralizing antibody, the neutralizing anti-RSV-F antibody may be 9C5.

Another aspect includes methods for producing a population of respiratory syncytial virus F polypeptide virus-like particles, comprising: (a) providing an expression vector which expresses a respiratory syncytial virus F polypeptide; (b) introducing the expression vector into a mammalian cell in a media; and (c) expressing the respiratory syncytial virus F polypeptide to produce the respiratory syncytial virus F polypeptide virus-like particles, wherein the virus-like particle may include one or more of the following characteristics: the virus-like particles do not comprise an enveloped virus core; the virus-like particles are pleomorphic, of non-uniform size, or of non-uniform shape; and/or the virus-like particles do not comprise an enveloped virus core forming polypeptide.

In another embodiment which may be combined with the preceding aspect, the method further includes the step of recovering the respiratory syncytial virus F polypeptide virus-like particles from the media in which the mammalian cell is cultured.

In another embodiment which may be combined with the preceding embodiment and aspect, the expression vector may be a viral vector. In another embodiment which may be combined with the preceding embodiments and aspect, the viral vector may be selected from the group consisting of: an adenovirus, a herpesvirus, a poxvirus and a retrovirus. In another embodiment which may be combined with the preceding embodiment and aspect, the mammalian cell may be selected from the group consisting of a BHK cell, a VERO cell, an HT1080 cell, an MRC-5 cell, a WI 38 cell, an MDCK cell, an MDBK cell, a 293 cell, a 293T cell, an RD cell, a COS-7 cell, a CHO cell, a Jurkat cell, a HUT cell, a SUPT cell, a C8166 cell, a MOLT4/clone8 cell, an MT-2 cell, an MT-4 cell, an H9 cell, a PM1 cell, a CEM cell, a myeloma cell, SB20 cell, a LtK cell, a HeLa cell, a WI-38 cell, an L2 cell, a CMT-93, and a CEMX174 cell.

In another embodiment which may be combined with the preceding embodiment and aspect, a neutralizing anti-RSV-F antibody may bind to the expressed respiratory syncytial virus F polypeptide (demonstrating that the respiratory syncytial virus F polypeptide is substantially in a native fold). In another embodiment which may be combined with the preceding embodiments and aspect including a neutralizing antibody, the neutralizing anti-RSV-F antibody is 9C5.

Another aspect includes methods for treating or preventing respiratory syncytial virus infection comprising administering to a subject an immunogenic amount of the preparation of any of the preceding embodiments of that aspect of the invention or the population produced by the method or any of the preceding embodiments of that aspect of the invention. Another embodiment that may be combined with any of the embodiments of the preceding aspect, the administration induces a protective immunization response in the subject. Another embodiment that may be combined with any of the embodiments of the preceding aspect, the administration may be selected from the group consisting of subcutaneous delivery, transcutaneous delivery, intradermal delivery, subdermal delivery, intramuscular delivery, peroral delivery, oral delivery, intranasal delivery, buccal delivery, sublingual delivery, intraperitoneal delivery, intravaginal delivery, anal delivery and intracranial delivery.

Another aspect includes pharmaceutical compositions comprising an immunogenic amount of the preparation of any of the preceding embodiments of that aspect of the invention or the population produced by the method or any of the preceding embodiments of that aspect of the invention. Another embodiment that may be combined with any of the embodiments of the preceding aspect, the pharmaceutical composition further includes a pharmaceutically acceptable carrier.

Another aspect includes methods for providing protection against respiratory syncytial virus infection comprising administering to a subject an immunogenic amount of the preparation of any of the preceding embodiments of that aspect of the invention or the population produced by the method or any of the preceding embodiments of that aspect of the invention. Another embodiment that may be combined with any of the embodiments of the preceding aspect, the administration may be selected from the group consisting of subcutaneous delivery, transcutaneous delivery, intradermal delivery, subdermal delivery, intramuscular delivery, peroral delivery, oral delivery, intranasal delivery, buccal delivery, sublingual delivery, intraperitoneal delivery, intravaginal delivery, anal delivery and intracranial delivery.

The foregoing aspects and embodiments thereof may further be combined with any of the embodiments disclosed in the specification. Additional aspects of the invention may be found throughout the specification which may be included with any of foregoing embodiments and/or the additional embodiments disclosed in the specification

### SUMMARY OF THE FIGURES

FIG. 1 shows the plasmid map of p3.1-RSVFT.

FIG. 2 shows the plasmid map of p3.1-shFv1.

FIG. 3 shows the plasmid map of p3.1-shFv2.

FIG. 4 shows the plasmid map for p3.1-Gag.

FIG. 5 shows cytometric analysis of surface expression of RSV F on cells transfected with RSV F and Gag expression vectors. Non-transfected cells and cells transfected with p3.1-Gag alone exhibit background fluorescence levels. Cells transfected with RSV F expression vectors, with and without p3.1-Gag, show significant levels of fluorescence as a result of F detection by the 9C5 monoclonal antibody and fluorescent secondary antibody.

FIG. 14 shows detection of RSV F antigenic activity in the 100,000 x g pellets from the medium of cells transfected with RSV F genes with and without co-transfection with a Gag gene.

FIG. 7 shows representative sections of electron micrographs of VLPs harvested from the medium of 239-F cells transfected with p3.1-RSVFT.

FIG. 8 shows the plasmid map for pFB-shFv1.

FIG. 9 shows the sucrose density gradient of shFv1 VLPs expressed in insect cells.

FIG. 10 shows a representative electron micrograph of aggregated RSV F VLPs and baculovirus particles in fraction 9 of the shFv1 sucrose gradient shown in FIG. 9.

FIG. 11 shows the plasmid map of p3.1-F-GPI which encodes the chimeric F polypeptide ectodomain fused to the GPI anchor signal from human carboxypeptidase M.

FIG. 12 shows flow cytometric analysis of surface expression of the modified F-GPI protein on cells transfected with p3.1-F-GPI with and without co-transfection with p3.1-Gag. Cells transfected with p3.1-Gag alone exhibited background fluorescence levels. Cells transfected with p3.1-F-GPI, with and without p3.1-Gag, showed significant levels of fluorescence as a result of F detection by the 9C5 monoclonal antibody and fluorescent secondary antibody.

FIG. 13 shows the western blot analysis of 100,000 x g pellets from the medium of cells transfected with p3.1-Gag, p3.1-F-GPI, and p3.1-Gag+p3.1-F-GPI. The lanes are from left to right: G, sample from p3.1-Gag transfection; F, sample from p3.1-F-GPI transfection; F+G, sample from the p3.1-Gag+p3.1-F-GPI transfection. Data show that F expression interferes with Gag budding, but Gag expression does not interfere with F budding.

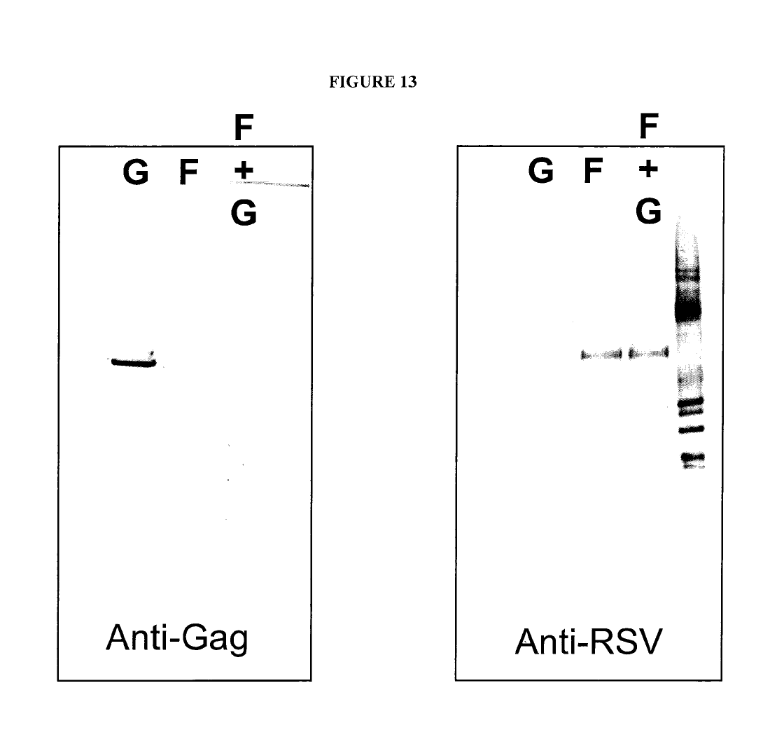

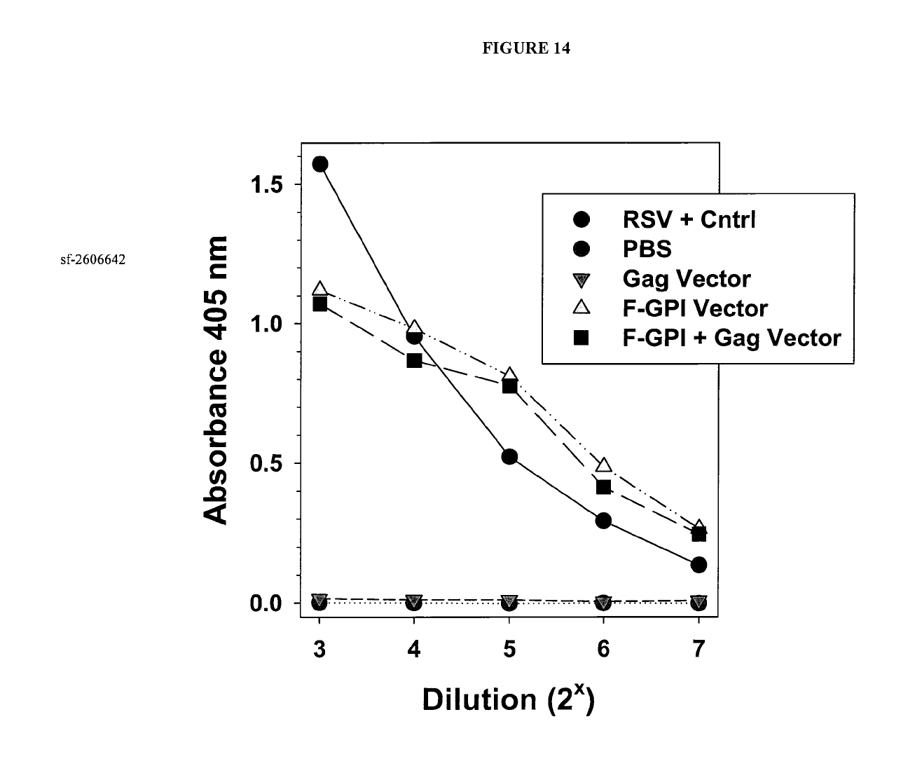

FIG. 14 sows detection of RSV F antigenic activity in the 100,000×g pellets from the medium of cells transfected with the RSV F-GPI vector with and without co-transfection with the Gag gene vector. A 100,000×g pellet of the medium from RSV-infected cells containing live RSV virion particles was included as a positive control demonstrating that F-GPI particles are antigenically similar to live RSV in terms of the F antigen.

### DETAILED DESCRIPTION OF THE PREFERRED EMBODIMENTS

Preferred embodiments of the present invention include, without limitation, respiratory syncytial virus F polypeptide VLPs preparations that preferably have been produced in a mammalian cell expression system; methods of expressing or generating such respiratory syncytial virus F polypeptide VLPs preparations; methods of further processing such preparations into vaccine compositions and methods of using such vaccine compositions.

Certain aspects and embodiments of the invention are based upon the surprising discovery that the respiratory syncytial virus F polypeptide by itself is sufficient to generate VLPs when expressed in an appropriate host cell in the absence of any core or core forming proteins such as a gag or matrix polypeptide. Such VLPs will have a lipid or envelope without the internal protein core or capsid. Therefore, such VLPs will have one or more of the following physical characteristics: no enveloped virus core, capsid or nucleocapsid within the lipid membrane or envelope; pleomorphic or non-uniform size and/or shape; the VLPs are not aggregated with one another; the VLPs are not associated or otherwise aggregated with a viral vector used to produce the VLPs, and mammalian glycosylation and/or mammalian membrane bound or associated proteins.

A preferred method of generating the respiratory syncytial virus F polypeptide is by expression in mammalian cells, preferably including coexpression of additional polypeptide antigens.

The practice of the disclosed methods and protocols will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press.

### Definitions

A “respiratory syncytial virus F polypeptide VLP” as used here refers to virus-like particles that are formed using a respiratory syncytial virus F polypeptide preferably expressed in the absence of any polypeptide capable of forming a viral capsid, nucleocapsid or core. Preferred examples include, without limitation, VLPs generated using an respiratory syncytial virus F polypeptide substantially lacking the cytoplasmic portion of the respiratory syncytial virus F polypeptide or an respiratory syncytial virus F polypeptide and VLPs generated using influenza M1 polypeptides and/or hemagglutinin polypeptides (and optionally neuraminidase polypeptides).

“Mammalian glycosylation” refers to glycosylation patterns generated by mammalian cell expression systems. Such glycosylation patterns does not include glycosylation patterns produced by insect cells that have been modified to include mammalian glycosylation enzymes, so long as such modified insect cells only produce “mammalian-like” glycosylation rather than the glycosylation pattern that would be naturally produced by a mammal or a mammalian cell based expression system. Preferred examples of mammalian glycosylation patterns include glycosylation produced by expression in human (e.g., HEK 293, HeLa), Chinese hamster ovary (CHO), dog (e.g., MDCK), mouse (e.g., H9), rat (e.g., IE) and non-human primate (e.g., NCTC) cells.

An “enveloped virus core forming polypeptide” as used herein includes any viral polypeptide or combination of viral polypeptides that are capable of forming a viral capsid, nucleocapsid or core within the envelope of an enveloped virus. Preferred embodiments of the respiratory syncytial virus F polypeptide VLPs described herein exclude enveloped virus core forming polypeptides as such polypeptides can interfere with the formation of VLPs by the respiratory syncytial virus F polypeptide. Polypeptides derived from enveloped virus core forming polypeptides that are not capable of forming a viral capsid, nucleocapsid or core or subsets of enveloped virus core forming polypeptides that are not capable of forming a viral capsid, nucleocapsid or core may be included in the VLPs disclosed herein as such polypeptides would not interfere with the formation of the VLPs disclosed herein.

Gag polypeptides, the retrovirus-derived structural polypeptide that is responsible for formation of the virus-like particles described herein, are examples enveloped virus core forming polypeptide. The genome of retroviruses codes for three major gene products: the gag gene coding for structural proteins, the pol gene coding for reverse transcriptase and associated proteolytic polypeptides, nuclease and integrase associated functions, and env whose encoded glycoprotein membrane proteins are detected on the surface of infected cells and also on the surface of mature released virus particles. The gag genes of all retroviruses have an overall structural similarity and within each group of retroviruses are conserved at the amino acid level. The gag gene gives rise to the core proteins excluding the reverse transcriptase.

For MLV the Gag precursor polyprotein is Pr65^{Gag} and is cleaved into four proteins whose order on the precursor is NH₂-p15-pp12-p30-p10-COOH. These cleavages are mediated by a viral protease and may occur before or after viral release depending upon the virus. The MLV Gag protein exists in a glycosylated and a non-glycosylated form. The glycosylated forms are cleaved from gPr80^{Gag} which is synthesized from a different inframe initiation codon located upstream from the AUG codon for the non-glycosylated Pr65^{Gag}. Deletion mutants of MLV that do not synthesize the glycosylated Gag are still infectious and the non-glycosylated Gag can still form virus-like particles, thus raising the question over the importance of the glycosylation events. The post translational cleavage of the HIV-1 Gag precursor of pr55^{Gag} by the virus coded protease yields the N-myristoylated and internally phosphorylated p17 matrix protein (p17MA), the phosphorylated p24 capsid protein (p24CA), and the nucleocapsid protein p15 (p15NC), which is further cleaved into p9 and p6.

Structurally, the prototypical Gag polyprotein is divided into three main proteins that always occur in the same order in retroviral gag genes: the matrix protein (MA) (not to be confused with influenza matrix protein Ml, which shares the name matrix but is a distinct protein from MA), the capsid protein (CA), and the nucleocapsid protein (NC). Processing of the Gag polyprotein into the mature proteins is catalyzed by the retroviral encoded protease and occurs as the newly budded viral particles mature. Functionally, the Gag polyprotein is divided into three domains: the membrane binding domain, which targets the Gag polyprotein to the cellular membrane; the interaction domain which promotes Gag polymerization; and the late domain which facilitates release of nascent virions from the host cell. The form of the Gag protein that mediates assembly is the polyprotein. Thus, the assembly domains need not lie neatly within any of the cleavage products that form later. The state of the art is quite advanced regarding these important functional elements. See, e.g., Hansen et al. J. Virol 64, 5306-5316, 1990; Will et al., AIDS 5, 639-654, 1991; Wang et al. J. Virol. 72, 7950-7959, 1998; McDonnell et al., J. Mol. Biol. 279, 921-928, 1998; Schultz and Rein, J. Virol. 63, 2370-2372, 1989; Accola et al., J. Virol. 72, 2072-2078, 1998; Borsetti et al., J. Virol., 72, 9313-9317, 1998; Bowzard et al., J. Virol. 72, 9034-9044, 1998; Krishna et al., J. Virol. 72, 564-577, 1998; Wills et al., J. Virol. 68, 6605-6618, 1994; Xiang et al., J. Virol. 70, 5695-5700, 1996; Gamier et al., J. Virol. 73, 2309-2320, 1999.

As included within the scope of enveloped virus core forming polypeptides, the gag polypeptide shall at a minimum include the functional elements for formation of virus-like particles independently of the respiratory syncytial virus F polypeptide VLPs. If the functional elements that compete or interfere with the respiratory syncytial virus F polypeptide are omitted then such gag polypeptides are not enveloped virus core forming polypeptides.

Examples of retroviral sources for Gag polypeptides include murine leukemia virus, human immunodeficiency virus, Alpharetroviruses (such as the avian leucosis virus or the Rous sarcoma virus), Betaretroviruses (such as mouse mammary tumor virus, Jaagsiekte sheep retrovirus and Mason-Phizer monkey virus), Gammaretroviruses (such as murine leukemia virus, feline leukemia virus, reticuloendotheliosis virus and gibbon ape leukemia virus), Deltaretroviruses (such as human T-lymphotrophic virus and bovine leukemia virus), Epsilonretroviruses (such as walleye dermal sarcoma virus), or Lentiviruses (human immunodeficiency virus type 1, HIV-2, simian immunodeficiency virus, feline immunodeficiency virus, equine infectious anemia virus, and caprine arthritis encephalitis virus).

An “enveloped virus core” as used herein includes any proteinaceous core found inside the lipid membrane or envelope of an enveloped virus or a virus like particle generated by components of an enveloped virus. The proteinaceous core may comprise all or a portion of the capsid or nucleocapsid or the matrix.

The “lipid raft” as used herein refers to the cell membrane microdomain in which the gag polypeptide concentrates during the viral particle assembly process.

A “lipid raft-associated polypeptide” as used herein refers to any polypeptide that is directly or indirectly associated with a lipid raft excluding any enveloped virus core forming polypeptide. The particular lipid raft-associated polypeptide used in the invention will depend on the desired use of the VLP and the role of the lipid raft-associated polypeptide (e.g., attaching the extracellular portion of the respiratory syncytial virus F protein to the VLP or attaching one or more additional antigens or adjuvants to the VLP).

The lipid raft-associated polypeptide can be an integral membrane protein or a lipid raft-associating portion, a protein or portion thereof directly associated with the lipid raft via a protein modification which causes association with the membrane such as lipid modification, or a polypeptide with an indirect association with the lipid raft via a lipid raft-associated polypeptide.

Many proteins with lipid anchors associate with lipid rafts. Often short fragments of such proteins are sufficient for lipid attachment making such fragments ideal for lipid raft associate as the fragments can be readily attached to other proteins and polypeptide that may not themselves naturally associate with lipid rafts. Lipid anchors that couple polypeptides to lipid rafts include GPI anchors, myristoylation, palmitoylation, and double acetylation.

Many different types of polypeptides are associated with lipid rafts. Lipid rafts function as platforms for numerous biological activities including signal transduction, membrane trafficking, viral entry, viral assembly, and budding of assembled particles and are therefore associated with the various polypeptides involved in these processes.

The various types of polypeptides involved in signaling cascades are associated with lipid rafts that function as signaling platforms. One type of lipid raft which functions as signaling platform is called a caveolae. It is a flask shaped invagination of the plasma-membrane which contains polypeptides from the caveolin family (e.g., caveolin and/or flottillin).

Membrane trafficking polypeptides are associated with lipid rafts which function as membrane trafficking platforms. Examples include the proteins involved in endocytosis and excocytosis, such as syntaxin-1, syntaxin-4, synapsin I, adducin, VAMP2, VAMP/synaptobrevin, synaptobrevin II, SNARE proteins, SNAP-25, SNAP-23, synaptotagmin I, synaptotagmin II, and the like.

Viral receptors, receptor-coreceptor complexes, any other components which help modulate the entry process are associated with lipid rafts which function as specialized membrane trafficking platforms for viral entry. Examples of lipid raft-associated viral receptors include the decay accelerating factor (DAF or CD55), a GPI-anchored membrane glycoprotein that is a receptor for many enteroviruses; the receptor for group A rotaviruses, a complex containing multiple components including gangliosides, Hsc70 protein, alpha2-betal and alpha5-beta2 integrins; glycoproteins of several enveloped viruses like HIV, MLV, measles, and Ebola; and polypeptides involved in HIV entry like CD5, CCR5, and nef. See Chazal and Gerlier, 2003, Virus Entry, Assembly, Budding, and Membrane Rafts, Microbiol. & Mol. Bio. Rev. 67(2):226-237.

Polypeptides involved in viral particle assembly are associated with lipid rafts functioning as viral assembly platforms. So long as portions that are responsible for formation of the viral nucleocapsid, capsid or core are omitted, such polypeptides or portions thereof may be used as lipid raft-associated polypeptides. Examples of such polypeptides include the HA and NA influenza envelope glycoproteins, the H and mature Fl-F2 fusion proteins from measles, and the gp160, gp41, and Pr55gag from HIV. See Chazal and Gerlier, 2003, Virus Entry, Assembly, Budding, and Membrane Rafts, Microbiol. And Mol. Bio. Rev. 67(2):226-237.

Polypeptides involved in budding of assembled virus are associated with lipid rafts that function as viral budding platforms. There is data suggesting that HIV-1 budding from the host cell occurs in membrane rafts. See Chazal and Gerlier, 2003, Virus Entry, Assembly, Budding, and Membrane Rafts, Microbiol. And Mol. Bio. Rev. 67(2):226-237. General information about polypeptides involved in viral budding can be found in Fields Virology (4th ed.) 2001.

Preferred lipid-raft associated polypeptides include viral polypeptides such as hemagglutinin polypeptide, neuraminidase polypeptide, fusion protein polypeptide, glycoprotein polypeptide, and envelope protein polypeptide. Each of these polypeptide can be from any type of virus; however, certain embodiments include envelope protein from HIV-1 virus, fusion protein from respiratory syncytial virus or measles virus, glycoprotein from respiratory syncytial virus, herpes simplex virus, or Ebola virus, and hemagglutinin protein from measles virus.

Preferred non-viral pathogen lipid-raft associated polypeptides may be obtained from pathogenic protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like. Such non-viral lipid-raft associated polypeptides may be used without being liked to an antigen not naturally associated with a lipid-raft as the lipid raft-associated polypeptide itself will act as the antigen.

A preferred example of a viral lipid-raft associated polypeptide is a hemagglutinin polypeptide. The “hemagglutinin polypeptide” as used herein is derived from the influenza virus protein that mediates binding of the virus to the cell to be infected. Hemagglutinin polypeptides may also be derived from the comparable measles virus protein. The protein is an antigenic glycoprotein found anchored to the surface of influenza viruses by a single membrane spanning domain. At least sixteen subtypes of the influenza hemagglutinin have been identified labeled H1 through H16. H1, H2, and H3, are found in human influenza viruses. Highly pathogenic avian flu viruses with H5 or H7 hemagglutinins have been found to infect humans at a low rate. It has been reported that single amino acid changes in the avian virus strain's type H5 hemagglutinin have been found in human patients that alters the receptor specificity to allow the H5 hemagglutinin to significantly alter receptor specificity of avian H5N1 viruses, providing them with an ability to bind to human receptors (109 and 110). This finding explains how an H5N1 virus that normally does not infect humans can mutate and become able to efficiently infect human cells.

Hemagglutinin is a homotrimeric integral membrane polypeptide. The membrane spanning domain naturally associates with the raft-lipid domains, which allows it to associate with the respiratory syncytial virus F polypeptides for incorporation into VLPs. It is shaped like a cylinder, and is approximately 135 Å long. The three identical monomers that constitute HA form a central coiled-coil and a spherical head that contains the sialic acid binding sites, which is exposed on the surface of the VLPs. HA monomers are synthesized as a single polypeptide precursor that is glycosylated and cleaved into two smaller polypeptides: the HA1 and HA2 subunits. The HA2 subunits form the trimeric coiled-coil that is anchored to the membrane and the HA1 subunits form the spherical head.

As used in certain VLPs of the present invention as a lipid-raft associated polyeptide, the hemagglutinin polypeptide shall at a minimum include the membrane anchor domain. The hemagglutinin polypeptide may be derived from any influenza virus type, subtype, strain or substrain, preferable from the H1, H2, H3, H5, H7, and H9 hemagglutinins. In addition, the hemagglutinin polypeptide may be a chimera of different influenza hemagglutinins. The hemagglutinin polypeptide preferably includes one or more additional antigens not naturally associated with a lipid raft that may be generated by splicing the coding sequence for the one or more additional polypeptides into the hemagglutinin polypeptide coding sequence. A preferred site for insertion of additional polypeptides into the hemagglutinin polypeptide is the N-terminus.

Another preferred example of a viral lipid-raft associated polypeptide is a neuraminidase polypeptide. The “neuraminidase polypeptide” as used herein is derived from the influenza virus protein that mediates release of the influenza virus from the cell by cleavage of terminal sialic acid residues from glycoproteins. The neuraminidase glycoprotein is expressed on the viral surface. The neuraminidase proteins are tetrameric and share a common structure consisting of a globular head with a beta-pinwheel structure, a thin stalk region, and a small hydrophobic region that anchors the protein in the virus membrane by a single membrane spanning domain. The active site for sialic acid residue cleavage includes a pocket on the surface of each subunit formed by fifteen charged amino acids, which are conserved in all influenza A viruses. At least nine subtypes of the influenza neuraminidase have been identified labeled N1 through N9.

As used in certain VLPs of the present invention, the neuraminidase polypeptide shall at a minimum include the membrane anchor domain. The state of the art regarding functional regions is quite high. See, e.g., Varghese et al., Nature 303, 35-40, 1983; Colman et al., Nature 303, 41-44, 1983; Lentz et al., Biochem, 26, 5321-5385, 1987; Webster et al., Virol. 135, 30-42, 1984. The neuraminidase polypeptide may be derived from any influenza virus type, subtype strain or substrain, preferably from the N1 and N2 neuraminidases. In addition, the neuraminidase polypeptide may be a chimera of different influenza neuraminidase. The neuraminidase polypeptide preferably includes one or more additional antigens that are not naturally associated with a lipid raft that may be generated by splicing the coding sequence for the one or more additional polypeptides into the hemagglutinin polypeptide. A preferred site for insertion of additional polypeptides into the neuraminidase polypeptide coding sequence is the C-terminus

Another preferred example of a lipid raft associated peptide is an insect derived adhesion protein termed fasciclin I (FasI). The “fasciclin I polypeptide” as used herein is derived from the insect protein that is involved in embryonic development. This non-viral protein can be expressed in an insect cell baculovirus expression system leading to lipid raft association of FasI (J. Virol. 77, 6265-6273, 2003). It therefore follows that attachment of a heterologous antigen to a fasciclin I polypeptide will lead to incorporation of the chimeric molecule into VLPs when co-expressed with respiratory syncytial F polypeptides. As used in the VLPs of the present invention, the fasciclin I polypeptide shall at a minimum include the membrane anchor domain.

Another preferred example of a lipid raft associated peptide is a viral derived attachment protein from RSV named the G glycoprotein. The “G glycopolypeptide” as used herein is derived from the RSV G glycoprotein. Recent data has demonstrated that lipid raft domains are important for RSV particle budding as they are for influenza virus (Virol 327, 175-185, 2004; Arch. Virol. 149, 199-210, 2004; Virol. 300, 244-254, 2002). The G glycoprotein from RSV is a 32.5 kd integral membrane protein that serves as a viral attachment protein as well as a protective antigen for RSV infection. As with the hemagglutinin from influenza virus, its antigenicity may enhance the antigenicity of any non-lipid raft antigens attached to it. Any modifications to the G glycopolypeptide in the way of non-lipid raft foreign antigen attachment will result in respiratory syncytial virus F polypeptide VLPs capable of inducing significant immune responses to the foreign antigen.

The terms “respiratory syncytial virus F polypeptide virus-like particle,” “respiratory syncytial virus F polypeptide VLP,” and “VLP” are used interchangeably throughout except where VLP by its context is referring to a virus-like particle that is not based upon an enveloped based virus or is based upon a particular component of certain enveloped-based viruses as disclosed herein.

### Antigens

Certain aspects of the present invention include additional antigens associated with the respiratory syncytial virus F polypeptide VLP preparations. Such additional antigens may be included in the same composition and may further be covalently or non-covalently associated with the VLPs. In preferred embodiments, the respiratory syncytial virus F polypeptide and/or other lipid raft-associated polypeptides are a readily adaptable platform for forming respiratory syncytial virus F polypeptide VLPs containing antigens which may not be naturally associated with a lipid raft. This section describes preferred antigens for use with the disclosed VLPs.

### Linkage Between Antigen and Lipid Raft-Associated Polypeptide

As a means for forming VLPs containing antigens not naturally associated with a lipid raft, a linkage may be formed between the respiratory syncytial virus F polypeptide and/or another lipid raft-associated polypeptide and the antigen. The lipid-raft associated polypeptide may be linked to a single antigen or to multiple antigens to increase immunogenicity of the VLP, to confer immunogenicity to various pathogens, or to confer immunogenicity to various strains of a particular pathogen.

The linkage between the antigen and a lipid raft-associated polypeptide can be any type of linkage sufficient to result in the antigen being incorporated into the VLP. The bond can be a covalent bond, an ionic interaction, a hydrogen bond, an ionic bond, a van der Waals force, a metal-ligand interaction, or an antibody-antigen interaction. In preferred embodiments, the linkage is a covalent bond, such as a peptide bond, carbon-oxygen bond, a carbon-sulfur bond, a carbon-nitrogen bond, a carbon-carbon bond, or a disulfide bond.

The antigen may be produced recombinantly with an existing linkage to the lipid-raft associated polypeptide or it may be produced as an isolated substance and then linked at a later time to the lipid-raft associated polypeptide.

### Antigen Types

The antigens as used herein can be any substance capable of eliciting an immune response and which does not naturally associate with a lipid raft. Antigens include, but are not limited to, proteins, polypeptides (including active proteins and individual polypeptide epitopes within proteins), glycopolypeptides, lipopolypeptides, peptides, polysaccharides, polysaccharide conjugates, peptide and non-peptide mimics of polysaccharides and other molecules, small molecules, lipids, glycolipids, and carbohydrates. If the antigen does not naturally associate either directly or indirectly with a lipid raft, it would not be expected to be incorporated into a VLP without linkage to a lipid raft-associated polypeptide. The antigen can be any antigen implicated in a disease or disorder, e.g., microbial antigens (e.g., viral antigens, bacterial antigens, fungal antigens, protozoan antigens, helminth antigens, yeast antigens, etc.), tumor antigens, allergens and the like.

### Sources for Antigens

The antigens described herein may be synthesized chemically or enzymatically, produced recombinantly, isolated from a natural source, or a combination of the foregoing. The antigen may be purified, partially purified, or a crude extract.

Polypeptide antigens may be isolated from natural sources using standard methods of protein purification known in the art, including, but not limited to, liquid chromatography (e.g., high performance liquid chromatography, fast protein liquid chromatography, etc.), size exclusion chromatography, gel electrophoresis (including one-dimensional gel electrophoresis, two-dimensional gel electrophoresis), affinity chromatography, or other purification technique. In many embodiments, the antigen is a purified antigen, e.g., from about 50% to about 75% pure, from about 75% to about 85% pure, from about 85% to about 90% pure, from about 90% to about 95% pure, from about 95% to about 98% pure, from about 98% to about 99% pure, or greater than 99% pure.

One may employ solid phase peptide synthesis techniques, where such techniques are known to those of skill in the art. See Jones, The Chemical Synthesis of Peptides (Clarendon Press, Oxford) (1994). Generally, in such methods a peptide is produced through the sequential additional of activated monomeric units to a solid phase bound growing peptide chain.

Well-established recombinant DNA techniques can be employed for production of polypeptides either in the same vector as the lipid-raft associated polypeptide, where, e.g., an expression construct comprising a nucleotide sequence encoding a polypeptide is introduced into an appropriate host cell (e.g., a eukaryotic host cell grown as a unicellular entity in in vitro cell culture, e.g., a yeast cell, an insect cell, a mammalian cell, etc.) or a prokaryotic cell (e.g., grown in in vitro cell culture), generating a genetically modified host cell; under appropriate culture conditions, the protein is produced by the genetically modified host cell.

### Viral Antigens

Suitable viral antigens include those associated with (e.g., synthesized by) viruses of one or more of the following groups: Retroviridae (e.g. human immunodeficiency viruses, such as HIV-1 (also referred to as HTLV-III, LAV or HTLV-III/LAV, or HIV-III); and other isolates, such as HIV-LP; Picomaviridae (e.g. polio viruses, hepatitis A virus; enteroviruses, human Coxsackie viruses, rhinoviruses, echoviruses); Calciviridae (e.g. strains that cause gastroenteritis, including Norwalk and related viruses); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); and astroviruses.

### Norvirus Antigens

The VLPs disclosed herein may preferably include various antigens from the Norovirus family Noroviruses, also called “Norwalk-like viruses” represent one of four genera within the Caliciviridae virus family Within the Norovirus genus there are two major genetic groups that have been designated Genogroup I and Genogroup II. Genogroup I Norovirus strains include Norwalk virus, Southampton virus, Desert Shield virus, and Chiba virus. Genogroup II Norovirus strains include Houston virus, Hawaii virus, Lordsdale virus, Grimsby virus, Mexico virus, and the Snow Mountain agent (Parker, T. D., et al. J Virol. (2005) 79(12):7402-9; Hale, A.D., et al. J Clin. Micro. (2000) 38(4):1656-1660). Norwalk virus (NV) is the prototype strain of a group of human caliciviruses responsible for the majority of epidemic outbreaks of acute viral gastroenteritis worldwide. The Norwalk virus capsid protein has two domains: the shell domain (S) and the protruding domain (P). The P domain (aa 226-530, Norwalk strain numbering) is divided into two subdomains, P1 and P2. The P2 domain is a 127 aa insertion (aa 279-405) in the P1 domain and is located at the most distal surface of the folded monomer. The P2 domain is the least conserved region of VP1 among norovirus strains, and the hypervariable region within P2 is thought to play an important role in receptor binding and immune reactivity. Given the external location of the P domain, it is the preferred antigen or source of polypeptide epitopes for use as antigens for the VLP vaccines disclosed herein. The P2 domain is a preferred antigen for Genogroup I or Genogroup II Norovirus strains. Even more preferred is the mAb 61.21 epitope recently identified as lying in a region of the P2 domain conserved across a range of norovirus strains, as well as the mAb 54.6 epitope (Lochridge, V.P., et al. J Gen. Virol. (2005) 86:2799-2806).

### Influenza Antigens

The VLPs disclosed herein may include various antigens from influenza including, without limitation, hemagglutinin, neuraminidase, or an additional influenza antigen. A preferred additional influenza antigen is the M2 polypeptide. The M2 polypeptide of influenza virus is a small 97 amino acid class III integral membrane protein encoded by RNA segment 7 (matrix segment) following a splicing event (80, 81). Very little M2 exists on virus particles but it can be found more abundantly on infected cells. M2 serves as a proton-selective ion channel that is necessary for viral entry (82, 83). It is minimally immunogenic during infection or conventional vaccination, explaining its conservation, but when presented in an alternative format it is more immunogenic and protective (84-86). This is consistent with observations that passive transfer of an M2 monoclonal antibody in vivo accelerates viral clearance and results in protection (87). When the M2 external domain epitope is linked to HBV core particles as a fusion protein it is protective in mice via both parenteral and intranasal inoculation and is most immunogenic when three tandem copies are fused to the N-terminus of the core protein (88-90). This is consistent with other carrier-hapten data showing that increased epitope density increases immunogenicity (91).

For intranasal delivery of an M2 vaccine an adjuvant is required to achieve good protection and good results have been achieved with LTR192G (88, 90) and CTA1-DD (89). The peptide can also be chemically conjugated to a carrier such as KLH, or the outer membrane protein complex of N. meningitides, or human papilloma virus VLPs and is protective as a vaccine in mice and other animals (92, 93).

Insofar as the M2 protein is highly conserved it is not completely without sequence divergence. The M2 ectodomain epitopes of common strains A/PR/8/34 (H1N1) and A/Aichi/68 (H3N2) were shown to be immunologically cross reactive with all other modern sequenced human strains except for A/Hong Kong/156/97 (H5N1)(92). Examination of influenza database sequences also shows similar divergence in the M2 sequence of other more recent pathogenic H5N1 human isolates such as A/Vietnam/1203/04. This finding demonstrates that a successful H5-specific pandemic vaccine incorporating M2 epitopes will need to reflect the M2 sequences that are unique to the pathogenic avian strains rather than M2 sequences currently circulating in human H1 and H3 isolates.

Additional proteins from influenza virus (other than HA, NA and M2) may be included in the VLP vaccine either by co-expression or via linkage of all or part of the additional antigen to the respiratory syncytial virus F polypeptide or other lipid raft-associated polypeptides. These additional antigens include PB2, PB1, PA, nucleoprotein, matrix (M1), NS1, and NS2. These latter antigens are not generally targets of neutralizing antibody responses but may contain important epitopes recognized by T cells. T cell responses induced by a VLP vaccine to such epitopes may prove beneficial in boosting protective immunity.

### Other Pathogenic Antigens

Suitable bacterial antigens include antigens associated with (e.g., synthesized by and endogenous to) any of a variety of pathogenic bacteria, including, e.g., pathogenic gram positive bacteria such as pathogenic Pasteurella species, Staphylococci species, and Streptococcus species; and gram-negative pathogens such as those of the genera Neisseria, Escherichia, Bordetella, Campylobacter, Legionella, Pseudomonas, Shigella, Vibrio, Yersinia, Salmonella, Haemophilus, Brucella, Francisella and Bacterioides. See, e.g., Schaechter, M, H. Medoff, D. Schlesinger, Mechanisms of Microbial Disease. Williams and Wilkins, Baltimore (1989)).

Suitable antigens associated with (e.g., synthesized by and endogenous to) infectious pathogenic fungi include antigens associated with infectious fungi including but not limited to: Cryptococcus neoformans, Histoplasma capsulatum, Coccidioides immitis, Blastomyces dermatitidis, and Candida albicans, Candida glabrata, Aspergillus fumigata, Aspergillus flavus, and Sporothrix schenckii.

Suitable antigens associated with (e.g., synthesized by and endogenous to) pathogenic protozoa, helminths, and other eukaryotic microbial pathogens include antigens associated with protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

Suitable antigens include antigens associated with (e.g., synthesized by and endogenous to) pathogenic microorganisms such as: Helicobacter pyloris, Borelia burgdorferi, Legionella pneumophila, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Chlamydia trachomatis, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israeli. Non-limiting examples of pathogenic E. coli strains are: ATCC No. 31618, 23505, 43886, 43892, 35401, 43896, 33985, 31619 and 31617.

Any of a variety of polypeptides or other antigens associated with intracellular pathogens may be included in the VLPs. Polypeptides and peptide epitopes associated with intracellular pathogens are any polypeptide associated with (e.g., encoded by) an intracellular pathogen, fragments of which are displayed together with MHC Class I molecule on the surface of the infected cell such that they are recognized by, e.g., bound by a T-cell antigen receptor on the surface of, a CD8.sup.+ lymphocyte. Polypeptides and peptide epitopes associated with intracellular pathogens are known in the art and include, but are not limited to, antigens associated with human immunodeficiency virus, e.g., HIV gp120, or an antigenic fragment thereof; cytomegalovirus antigens; Mycobacterium antigens (e.g., Mycobacterium avium, Mycobacterium tuberculosis, and the like); Pneumocystic carinii (PCP) antigens; malarial antigens, including, but not limited to, antigens associated with Plasmodium falciparum or any other malarial species, such as 41-3, AMA-1, CSP, PFEMP-1, GBP-130, MSP-1, PFS-16, SERP, etc.; fungal antigens; yeast antigens (e.g., an antigen of a Candida spp.); toxoplasma antigens, including, but not limited to, antigens associated with Toxoplasma gondii, Toxoplasma encephalitis, or any other Toxoplasma species; Epstein-Barr virus (EBV) antigens; Plasmodium antigens (e.g., gp190/MSP1, and the like); etc.

A preferred VLP vaccine may be directed against Bacillus anthracis. Bacillus anthracis are aerobic or facultative anaerobic Gram-positive, nonmotile rods measuring 1.0 μm wide by 3.0-5.0 μm long. Under adverse conditions, B. anthracis form highly resistant endospores, which can be found in soil at sites where infected animals previously died. A preferred antigen for use in a VLP vaccine as disclosed herein is the protective antigen (PA), an 83 kDa protein that binds to receptors on mammalian cells and is critical to the ability of B. anthracis to cause disease. A more preferred antigen is the C-terminal 140 amino acid fragment of Bacillus anthracis PA which may be used to induce protective immunity in a subject against Bacillus anthracis. Other exemplary antigens for use in a VLP vaccine against anthrax are antigens from the anthrax spore (e.g., Bc1A), antigens from the vegetative stage of the bacterium (e.g., a cell wall antigen, capsule antigen (e.g., poly-gamma-D-glutamic acid or PGA), secreted antigen (e.g., exotoxin such as protective antigen, lethal factor, or edema factor). Another preferred antigen for use in a VLP vaccine is the tetra-saccharide containing anthrose, which is unique to B. anthracis (Daubenspeck J. M., et al. J. Biol. Chem. (2004), 279:30945). The tetra-saccharide may be coupled to a lipid raft-associated polypeptide allowing association of the antigen with the VLP vaccine.

### Tumor-Associated Antigens

Any of a variety of known tumor-specific antigens or tumor-associated antigens (TAA) can be included in the VLPs. The entire TAA may be, but need not be, used. Instead, a portion of a TAA, e.g., an epitope, may be used. Tumor-associated antigens (or epitope-containing fragments thereof) which may be used in VLPs include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV18, TUAN, alpha-feto protein (AFP), OFP, CA-125, CA-50, CA-19-9, renal tumor-associated antigen G250, EGP-40 (also known as EpCAM), S100 (malignant melanoma-associated antigen), p53, and p21ras. A synthetic analog of any TAA (or epitope thereof), including any of the foregoing, may be used. Furthermore, combinations of one or more TAAs (or epitopes thereof) may be included in the composition.

### Allergens

In one aspect, the antigen that is part of the VLP vaccine may be any of a variety of allergens. Allergen based vaccines may be used to induce tolerance in a subject to the allergen. Examples of an allergen vaccine involving co-precipitation with tyrosine may be found in U.S. Pat. Nos. 3,792,159, 4,070,455, and 6,440,426.

Any of a variety of allergens can be included in VLPs. Allergens include but are not limited to environmental aeroallergens; plant pollens such as ragweed/hayfever; weed pollen allergens; grass pollen allergens; Johnson grass; tree pollen allergens; ryegrass; arachnid allergens, such as house dust mite allergens (e.g., Der p I, Der f I, etc.); storage mite allergens; Japanese cedar pollen/hay fever; mold spore allergens; animal allergens (e.g., dog, guinea pig, hamster, gerbil, rat, mouse, etc., allergens); food allergens (e.g., allergens of crustaceans; nuts, such as peanuts; citrus fruits); insect allergens; venoms: (Hymenoptera, yellow jacket, honey bee, wasp, hornet, fire ant); other environmental insect allergens from cockroaches, fleas, mosquitoes, etc.; bacterial allergens such as streptococcal antigens; parasite allergens such as Ascaris antigen; viral antigens; fungal spores; drug allergens; antibiotics; penicillins and related compounds; other antibiotics; whole proteins such as hormones (insulin), enzymes (streptokinase); all drugs and their metabolites capable of acting as incomplete antigens or haptens; industrial chemicals and metabolites capable of acting as haptens and functioning as allergens (e.g., the acid anhydrides (such as trimellitic anhydride) and the isocyanates (such as toluene diisocyanate)); occupational allergens such as flour (e.g., allergens causing Baker's asthma), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates.

Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Altemaria (Altemaria altemata); Alder; Alnus (Alnus gultinoas); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherun elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

### Preferred Methods of Making RSV F VLPs from Mammalian Cells

Respiratory syncytial virus F polypeptide VLPs may be made by any method available to one of skill in the art. Respiratory syncytial virus F polypeptide VLPs include the respiratory syncytial virus F polypeptide which is responsible for the formation of the VLP while excluding any enveloped virus core forming polypeptides. In addition, the respiratory syncytial virus F polypeptide VLP may include one or more additional polypeptide such as a membrane (including lipid-raft)-associated polypeptide to provide (additional) antigens (other than those present naturally or artificially as a part of the one or more polypeptides responsible for the formation of the VLP). In preferred embodiments, the polypeptides may be co-expressed in any available protein expression system, preferably a mammalian cell-based system that includes lipid raft domains in the plasma membrane.

Recombinant expression of the polypeptides for the VLPs involves expression vectors containing polynucleotides that encode one or more of the polypeptides. Once a polynucleotide encoding one or more of the polypeptides has been obtained, the vector for the production of the polypeptide may be produced by recombinant DNA technology using techniques well known in the art. Thus, methods for preparing a protein by expressing a polynucleotide containing any of the VLP polypeptide-encoding nucleotide sequences are described herein. Methods which are well known to those skilled in the art can be used to construct expression vectors containing the VLP polypeptide coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding respiratory syncytial virus F polypeptide and optionally one or more additional lipid raft-associated polypeptides, all operably linked to one or more promoters.

Non-limiting examples of vectors that can be used to express sequences that assembly into VLPs as described herein include viral-based vectors (e.g., retrovirus, adenovirus, adeno-associated virus, lentivirus), plasmid vectors, non-viral vectors, mammalian vectors, mammalian artificial chromosomes (e.g., liposomes, particulate carriers, etc.) and combinations thereof.

The expression vector(s) typically contain(s) coding sequences and expression control elements which allow expression of the coding regions in a suitable host. The control elements generally include a promoter, enhancer, exon, intron, splicing sites translation initiation codon, and translation and transcription termination sequences, and an insertion site for introducing the insert into the vector. Translational control elements have been reviewed by M. Kozak (e.g., Kozak, M., Mamm. Genome 7(8):563-574, 1996; Kozak, M., Biochimie 76(9):815-821, 1994; Kozak, M., J Cell Biol 108(2):229-241, 1989; Kozak, M., and Shatkin, A. J., Methods Enzymol 60:360-375, 1979).

For example, typical promoters used for mammalian cell expression include the SV40 early promoter, a CMV promoter such as the CMV immediate early promoter (a CMV promoter can include intron A), RSV, HIV-LTR, the mouse mammary tumor virus LTR promoter (MMLV-LTR), FIV-LTR, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook, et al., supra, as well as a bovine growth hormone terminator sequence. Introns, containing splice donor and acceptor sites, may also be designed into the constructs as described herein (Chapman et al., Nuc. Acids Res. (1991) 19:3979-3986).

Enhancer elements may also be used herein to increase expression levels of the constructs, for example in mammalian host cells. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence (Chapman et al., Nuc. Acids Res. (1991) 19:3979-3986).

It will be apparent that a vector may contain one or more sequences as described herein. For example, a single vector may carry sequences encoding all the proteins found in the VLP. Alternatively, multiple vectors may be used (e.g., multiple constructs, each encoding a single polypeptide-encoding sequence or multiple constructs, each encoding one or more polypeptide-encoding sequences). In embodiments in which a single vector comprises multiple polypeptide-encoding sequences, the sequences may be operably linked to the same or different transcriptional control elements (e.g., promoters) within the same vector.

In addition, one or more sequences encoding non-RSV proteins may be expressed and incorporated into the VLP, including, but not limited to, sequences comprising and/or encoding immunomodulatory molecules (e.g., adjuvants described below), for example, immunomodulating oligonucleotides (e.g., CpGs), cytokines, detoxified bacterial toxins and the like.

The expression vector may be transferred to a host cell by conventional techniques and the transfected cells are then cultured by conventional techniques to produce the VLP polypeptide(s). Thus, the invention includes host cells containing a polynucleotide encoding one or more of the VLP polypeptides operably linked to a heterologous promoter. In preferred embodiments for the generation of VLPs, vectors encoding the respiratory syncytial virus F polypeptide and optionally a lipid-raft associated polypeptide linked to an antigen or adjuvant may be (co)expressed in the host cell for generation of the VLP, as detailed below.

Preferably, the VLPs are produced in eukaryotic cells, more preferably mammalian cells, following transfection, establishment of continuous cell lines (using standard protocols as known to one skilled in the art) and/or infection with DNA molecules that carry the RSV genes of interest. The level of expression of the proteins required for VLP formation is maximized by sequence optimization of the eukaryotic or viral promoters that drive transcription of the selected genes. The VLP is released into the culture medium driven by the respiratory syncytial virus F polypeptide, from where the VLP may be purified and subsequently formulated as vaccine. The VLPs are not infectious vaccines and therefore vaccine inactivation is not required.

The ability of respiratory syncytial virus F polypeptides expressed from sequences as described herein to self-assemble into VLPs with antigenic proteins presented on the surface allows these VLPs to be produced in any host cell by the co-introduction of the desired sequences. The sequence(s) (e.g., in one or more expression vectors) may be stably and/or transiently integrated in various combinations into a host cell.

Suitable host cells include, but are not limited to, bacterial, mammalian, baculovirus/insect, yeast, plant and Xenopus cells.

For example, a number of mammalian cell lines are known in the art and include primary cells as well as immortalized cell lines available from the American Type Culture Collection (A.T.C.C.), such as, but not limited to, BHK, VERO, HT1080, MRC-5, WI 38, MDCK, MDBK, 293, 293T, RD, COS-7, CHO, Jurkat, HUT, SUPT, C8166, MOLT4/clone8, MT-2, MT-4, H9, PM1, CEM, myeloma cells (e.g., SB20 cells), LtK, HeLa, WI-38, L2, CMT-93, and CEMX174 (such cell lines are available, for example, from the A.T.C.C.).

Similarly, bacterial hosts such as E. coli, Bacillus subtilis, and Streptococcus spp., will find use with the present expression constructs.

Yeast hosts useful in the present disclosure include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Fungal hosts include, for example, Aspergillus.

Insect cells for use with baculovirus expression vectors include, inter alia, Aedes aegypti, Autographa californica, Bombyx mori, Drosophila melanogaster, Spodoptera frugiperda, and Trichoplusia ni. See, Latham & Galarza (2001) J. Virol. 75(13):6154-6165; Galarza et al. (2005) Viral. Immunol. 18(1):244-51; and U.S. Patent Publications 200550186621 and 20060263804.

Cell lines expressing one or more of the sequences described above can readily be generated given the disclosure provided herein by stably integrating one or more sequences (expression vectors) encoding the respiratory syncytial virus F polypeptide of the VLP. The promoter regulating expression of the stably integrated respiratory syncytial virus F polypeptide sequences (s) may be constitutive or inducible.

The parent cell line from which a respiratory syncytial virus F polypeptide VLP-producer cell line is derived can be selected from any cell described above, including for example, mammalian, insect, yeast, bacterial cell lines. In a preferred embodiment, the cell line is a mammalian cell line (e.g., 293, RD, COS-7, CHO, BHK, VERO, MRC-5, HT1080, and myeloma cells). Production of RSV VLPs using mammalian cells provides (i) VLP formation; (ii) correct myristylation, glycosylation and budding; (iii) absence of non-mammalian cell contaminants and (iv) ease of purification.

In addition to creating cell lines, RSV-encoding sequences may also be transiently expressed in host cells. Suitable recombinant expression host cell systems include, but are not limited to, bacterial, mammalian, baculovirus/insect, vaccinia, Semliki Forest virus (SFV), Alphaviruses (such as, Sindbis, Venezuelan Equine Encephalitis (VEE)), mammalian, yeast and Xenopus expression systems, well known in the art. Particularly preferred expression systems are mammalian cell lines, vaccinia, Sindbis, insect and yeast systems.

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S, and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997).

When expression vectors containing the altered genes that code for the proteins required for VLP formation are introduced into host cell(s) and subsequently expressed at the necessary level, the VLP assembles and is then released from the cell surface into the culture media.

Depending on the expression system and host selected, the VLPs are produced by growing host cells transformed by an expression vector under conditions whereby the particle-forming polypeptides are expressed and VLPs can be formed. The selection of the appropriate growth conditions is within the skill of the art.

The particles are then isolated (or substantially purified) using methods that preserve the integrity thereof, such as, by density gradient centrifugation, e.g., sucrose gradients, PEG-precipitation, pelleting, and the like (see, e.g., Kimbauer et al. J. Virol. (1993) 67:6929-6936), as well as standard purification techniques including, e.g., ion exchange and gel filtration chromatography.

### Preferred Methods of Inactivation of Infectious Agents in Respiratory Syncytial Virus F Polypeptide VLPs VLP Preparations

A preferred method of inactivation is through electromagnetic radiation as electromagnetic radiation is capable of inactivating the infectious agents without substantially reducing the immunogenicity of the respiratory syncytial virus F polypeptide VLP. As all three preferred modes of electromagnetic radiation (i.e, UV irradiation with photoreactive compounds, UV irradiation alone and gamma irradiation) have a long history of use for inactivation of pathogens in a wide variety of samples such as blood, food, vaccines, etc. there are a wide variety of commercially available apparatus for applying the inactivating electromagnetic radiation that may be used with little to no modification to practice the methods disclosed herein. Furthermore, optimizing wavelengths and dosages is routine in the art and therefore readily within the capabilities of one of ordinary skill in the art.

### UV Irradiation with Photoreactive Compounds

An exemplary method of inactivation with electromagnetic radiation is a combination of ultraviolet irradiation, such as UV-A irradiation, in the presence of a photoreactive compound, preferable one that will react with polynucleotides in the infectious agent.

Preferred photoreactive compounds include: actinomycins, anthracyclinones, anthramycin, benzodipyrones, fluorenes, fluorenones, furocoumarins, mitomycin, monostral fast blue, norphillin A, phenanthridines, phenazathionium salts, phenazines, phenothiazines, phenylazides, quinolines, and thiaxanthenones. A preferred species are furocoumarins which belong in one of two main categories. The first category is psoralens [H-furo(3,2-g)-(1)-benzopyran-7-one, or delta-lactone of 6-hydroxy-5-benzofuranacrylic acid], which are linear and in which the two oxygen residues appended to the central aromatic moiety have a 1, 3 orientation, and further in which the furan ring moiety is linked to the 6 position of the two ring coumarin system. The second category is isopsoralens [2H-furo(2,3-h)-(1)-benzopyran-2-one, or delta-lactone of 4-hydroxy-5-benzofuranacrylic acid], which are angular and in which the two oxygen residues appended to the central aromatic moiety have a 1, 3 orientation, and further in which the furan ring moiety is linked to the 8 position of the two ring coumarin system. Psoralen derivatives may be generated by substitution of the linear furocoumarin at the 3, 4, 5, 8, 4′, or 5′ positions, while isopsoralen derivatives may be generated by substitution of the angular furocoumarin at the 3, 4, 5, 6, 4′, or 5 positions. Psoralens can intercalate between the base pairs of double-stranded nucleic acids, forming covalent adducts to pyrimidine bases upon absorption of long wave ultraviolet light (UVA). See, e.g., G. D. Cimino et al., Ann. Rev. Biochem. 54:1151 (1985); Hearst et al., Quart. Rev. Biophys. 17:1 (1984).

The wavelengths of the preferred UV (or in some cases visible light) radiation will depend upon the wavelength at which photoadducts are generated which is dependent upon the chemistry of the photoreactive chemical. By way of example, UV radiation in the wavelengths between 320 and 380 nm are most effective for many psoralens with 330 to 360 nm having maximum effectiveness.

### UV Irradiation Alone

In addition to UV irradiation in the presence of a photoreactive compound, infectious agents may be inactivated by UV irradiation alone. In a preferred embodiment, the radiation is UVC radiation having a wavelength between about 180 and 320 nm, or between about 225 and 290 nm, or about 254 nm (i.e., the maximal absorbance peak of polynucleotides). UVC radiation is preferred because it is less detrimental to the components of the respiratory syncytial virus F polypeptide VLPs disclosed herein for both stability and immunogenicity such as the lipid bilayer forming the envelope while retaining sufficient energy to inactivate infectious agents. However, other types of UV radiation such as, for example, UVA and UVB may also be used.

### Gamma Irradiation

Gamma irradiation (i.e., ionizing radiation) may also be used in the practice of the methods disclosed herein to generate the compositions. Gamma irradiation can inactivate infectious agents by introducing strand breaks in the polynucleotides encoding the genome of the infectious agent or by generating hydroxyl radicals that attack the polynucleotides.

### Preferred Methods of Using Respiratory Syncytial Virus F Polypeptide VLPs

### Formulations

A preferred use of the respiratory syncytial virus F polypeptide VLPs described herein is as a vaccine preparation. Typically, such vaccines are prepared as injectables either as liquid solutions or suspensions; solid forms suitable for solution in, or suspension in, liquid prior to injection may also be prepared. Such preparations may also be emulsified or produced as a dry powder. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain auxiliary substances such as wetting or emulsifying agents, pH buffering agents, or adjuvants which enhance the effectiveness of the vaccines.

Vaccines may be conventionally administered parenterally, by injection, for example, either subcutaneously, intradermally, subdermally or intramuscularly. Additional formulations which are suitable for other modes of administration include suppositories and, in some cases, oral, intranasal, buccal, sublingual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. In certain embodiments, a low melting wax, such as a mixture of fatty acid glycerides or cocoa butter is first melted and the respiratory syncytial virus F polypeptide VLPs described herein are dispersed homogeneously, for example, by stirring. The molten homogeneous mixture is then poured into conveniently sized molds, allowed to cool, and to solidify.

Formulations suitable for intranasal delivery include liquids and dry powders. Formulations include such normally employed excipients as, for example, pharmaceutical grades of mannitol, lactose, sucrose, trehalose, and chitosan. Mucosadhesive agents such as chitosan can be used in either liquid or powder formulations to delay mucocilliary clearance of intranasally-administered formulations. Sugars such as mannitol and sucrose can be used as stability agents in liquid formulations and as stability and bulking agents in dry powder formulations. In addition, adjuvants such as monophosphoryl lipid A (MPL) can be used in both liquid and dry powder formulations as an immunostimulatory adjuvant.

Formulations suitable for oral delivery include liquids, solids, semi-solids, gels, tablets, capsules, lozenges, and the like. Formulations suitable for oral delivery include tablets, lozenges, capsules, gels, liquids, food products, beverages, nutraceuticals, and the like. Formulations include such normally employed excipients as, for example, pharmaceutical grades of mannitol, lactose, starch, magnesium stearate, sodium saccharine, cellulose, magnesium carbonate, and the like. Other respiratory syncytial virus F polypeptide VLPs vaccine compositions may take the form of solutions, suspensions, pills, sustained release formulations or powders and contain 10-95% of active ingredient, preferably 25-70%. For oral formulations, cholera toxin is an interesting formulation partner (and also a possible conjugation partner).

The respiratory syncytial virus F polypeptide VLP vaccines when formulated for vaginal administration may be in the form of pessaries, tampons, creams, gels, pastes, foams or sprays. Any of the foregoing formulations may contain agents in addition to respiratory syncytial virus F polypeptide VLPs, such as carriers, known in the art to be appropriate.

In some embodiments, the respiratory syncytial virus F polypeptide VLP vaccine may be formulated for systemic or localized delivery. Such formulations are well known in the art. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Systemic and localized routes of administration include, e.g., intradermal, topical application, intravenous, intramuscular, etc.

The respiratory syncytial virus F polypeptide VLPs may be formulated into the vaccine including neutral or salt-based formulations. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines may be administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2000 μg (even though higher amounts in the 1-10 mg range are contemplated), such as in the range from about 0.5 μg to 1000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the vaccine formulations will be sufficiently immunogenic as a vaccine by themselves, but for some of the others the immune response will be enhanced if the vaccine further includes an adjuvant substance.

Delivery agents that improve mucoadhesion can also be used to improve delivery and immunogenicity especially for intranasal, oral or lung based delivery formulations. One such compound, chitosan, the N-deacetylated form of chitin, is used in many pharmaceutical formulations (32). It is an attractive mucoadhesive agent for intranasal vaccine delivery due to its ability to delay mucociliary clearance and allow more time for mucosal antigen uptake and processing (33, 34). In addition, it can transiently open tight junctions which may enhance transepithelial transport of antigen to the NALT. In a recent human trial, a trivalent inactivated influenza vaccine administered intranasally with chitosan but without any additional adjuvant yielded seroconversion and HI titers that were only marginally lower than those obtained following intramuscular inoculation (33).

Chitosan can also be formulated with adjuvants that function well intranasally such as the genetically detoxified E. coli heat-labile enterotoxin mutant LTK63. This adds an immunostimulatory effect on top of the delivery and adhesion benefits imparted by chitosan resulting in enhanced mucosal and systemic responses (35).

Finally, it should be noted that chitosan formulations can also be prepared in a dry powder format that has been shown to improve vaccine stability and result in a further delay in mucociliary clearance over liquid formulations (42). This was seen in a recent human clinical trial involving an intranasal dry powder diphtheria toxoid vaccine formulated with chitosan in which the intranasal route was as effective as the traditional intramuscular route with the added benefit of secretory IgA responses (43). The vaccine was also very well tolerated. Intranasal dry powdered vaccines for anthrax containing chitosan and MPL induce stronger responses in rabbits than intramuscular inoculation and are also protective against aerosol spore challenge (44).

Intranasal vaccines represent a preferred formulation as they can affect the upper and lower respiratory tracts in contrast to parenterally administered vaccines which are better at affecting the lower respiratory tract. This can be beneficial for inducing tolerance to allergen-based vaccines and inducing immunity for pathogen-based vaccines.

In addition to providing protection in both the upper and lower respiratory tracts, intranasal vaccines avoid the complications of needle inoculations and provide a means of inducing both mucosal and systemic humoral and cellular responses via interaction of particulate and/or soluble antigens with nasopharyngeal-associated lymphoid tissues (NALT) (16-19). The intranasal route has been historically less effective than parenteral inoculation, but the use of respiratory syncytial virus F polypeptide VLPs, novel delivery formulations, and adjuvants are beginning to change the paradigm. Indeed, respiratory syncytial virus F polypeptide VLPs containing functional hemagglutinin polypeptides may be especially well suited for intranasal delivery due to the abundance of sialic acid-containing receptors in the nasal mucosa resulting in the potential for enhanced HA antigen binding and reduced mucociliary clearance.

### Adjuvants

Various methods of achieving adjuvant effect for vaccines are known and may be used in conjunction with the respiratory syncytial virus F polypeptide VLPs disclosed herein. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9.

In some embodiments, a respiratory syncytial virus F polypeptide VLP vaccine includes the respiratory syncytial virus F polypeptide VLPs in admixture with at least one adjuvant, at a weight-based ratio of from about 10:1 to about 10¹⁰:1 respiratory syncytial virus F polypeptide VLP:adjuvant, e.g., from about 10:1 to about 100:1, from about 100:1 to about 10³:1, from about 10³:1 to about 10⁴:1, from about 10⁴:1 to about 10⁵:1, from about 10⁵:1 to about 10⁶:1, from about 10⁶:1 to about 10⁷:1, from about 10⁷:1 to about 10⁸:1, from about 10⁸:1 to about 10⁹:1, or from about 10⁹:1 to about 10¹⁰:1 respiratory syncytial virus F polypeptide VLP:adjuvant. One of skill in the art can readily determine the appropriate ratio through information regarding the adjuvant and routine experimentation to determine optimal ratios.

Preferred examples of adjuvants are polypeptide adjuvants that may be readily added to the respiratory syncytial virus F polypeptide VLPs described herein by co-expression with the polypeptide component of the respiratory syncytial virus F polypeptide VLP or fusion with the polypeptide component to produce chimeric polypeptides. Bacterial flagellin, the major protein constituent of flagella, is a preferred adjuvant which has received increasing attention as an adjuvant protein because of its recognition by the innate immune system by the toll-like receptor TLR5 (65). Flagellin signaling through TLR5 has effects on both innate and adaptive immune functions by inducing DC maturation and migration as well as activation of macrophages, neutrophils, and intestinal epithelial cells resulting in production of proinflammatory mediators (66-72).

TLR5 recognizes a conserved structure within flagellin monomers that is unique to this protein and is required for flagellar function, precluding its mutation in response to immunological pressure (73). The receptor is sensitive to a 100 fM concentration but does not recognize intact filaments. Flagellar disassembly into monomers is required for binding and stimulation.

As an adjuvant, flagellin has potent activity for induction of protective responses for heterologous antigens administered either parenterally or intranasally (66, 74-77) and adjuvant effects for DNA vaccines have also been reported (78). A Th2 bias is observed when flagellin is employed which would be appropriate for a respiratory virus such as influenza but no evidence for IgE induction in mice or monkeys has been observed. In addition, no local or systemic inflammatory responses have been reported following intranasal or systemic administration in monkeys (74). The Th2 character of responses elicited following use of flagellin is somewhat surprising since flagellin signals through TLR5 in a MyD88-dependent manner and all other MyD88-dependent signals through TLRs have been shown to result in a Th1 bias (67, 79). Importantly, pre-existing antibodies to flagellin have no appreciable effect on adjuvant efficacy (74) making it attractive as a multi-use adjuvant.

A common theme in many recent intranasal vaccine trials is the use of adjuvants and/or delivery systems to improve vaccine efficacy. In one such study an influenza H3 vaccine containing a genetically detoxified E. coli heat-labile enterotoxin adjuvant (LT R192G) resulted in heterosubtypic protection against H5 challenge but only following intranasal delivery. Protection was based on the induction of cross neutralizing antibodies and demonstrated important implications for the intranasal route in development of new vaccines (22).

Cytokines, colony-stimulating factors (e.g., GM-CSF, CSF, and the like); tumor necrosis factor; interleukin-2, -7, -12, interferons and other like growth factors, may also be used as adjuvants and are also preferred as they may be readily included in the respiratory syncytial virus F polypeptide VLP vaccine by admixing or fusion with the polypeptide component.

In some embodiments, the respiratory syncytial virus F polypeptide VLP vaccine compositions disclosed herein may include other adjuvants that act through a Toll-like receptor such as a nucleic acid TLR9 ligand comprising a 5′-TCG-3′ sequence; an imidazoquinoline TLR7 ligand; a substituted guanine TLR7/8 ligand; other TLR7 ligands such as Loxoribine, 7-deazadeoxyguanosine, 7-thia-8-oxodeoxyguanosine, Imiquimod (R-837), and Resiquimod (R-848).

Certain adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; MPL; and an encapsulating adjuvant.

Additional examples of adjuvants include agents such as aluminum salts such as hydroxide or phosphate (alum), commonly used as 0.05 to 0.1 percent solution in buffered saline (see, e.g., Nicklas (1992) Res. Immunol. 143:489-493), admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

DDA (dimethyldioctadecylammonium bromide) is an interesting candidate for an adjuvant, but also Freund's complete and incomplete adjuvants as well as quillaja saponins such as QuilA and QS21 are interesting. Further possibilities include poly[di(earboxylatophenoxy)phosphazene (PCPP) derivatives of lipopolysaccharides such as monophosphoryl lipid A (MPL®), muramyl dipeptide (MDP) and threonyl muramyl dipeptide (tMDP). The lipopolysaccharide based adjuvants are preferred for producing a predominantly Th1-type response including, for example, a combination of monophosphoryl lipid A, preferably 3-de-O-acylated monophosphoryl lipid A, together with an aluminum salt. MPL® adjuvants are available from GlaxoSmithKline (see, for example, U.S. Pat. Nos. 4,436,727; 4,877,611; 4,866,034 and 4,912,094).

Liposome formulations are also known to confer adjuvant effects, and therefore liposome adjuvants are preferred examples in conjunction with the respiratory syncytial virus F polypeptide VLPs.

Immunostimulating complex matrix type (ISCOM® matrix) adjuvants are preferred choices according to the invention, especially since it has been shown that this type of adjuvants are capable of up-regulating MHC Class II expression by APCs. An ISCOM matrix consists of (optionally fractionated) saponins (triterpenoids) from Quillaja saponaria, cholesterol, and phospholipid. When admixed with the immunogenic protein such as in the VLPs, the resulting particulate formulation is what is known as an ISCOM particle where the saponin may constitute 60-70% w/w, the cholesterol and phospholipid 10-15% w/w, and the protein 10-15% w/w. Details relating to composition and use of immunostimulating complexes can for example be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 provide useful instructions for the preparation of complete immunostimulating complexes.

The saponins, whether or not in the form of iscoms, that may be used in the adjuvant combinations with the respiratory syncytial virus F polypeptide VLP vaccines disclosed herein include those derived from the bark of Quillaja Saponaria Molina, termed Quil A, and fractions thereof, described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particularly preferred fractions of Quil A are QS21, QS7, and QS17.

β-Escin is another preferred haemolytic saponins for use in the adjuvant compositions of the present invention. Escin is described in the Merck index (12th ed: entry 3737) as a mixture of saponins occurring in the seed of the horse chestnut tree, Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin have been purified and shown to be biologically active (Yoshikawa M, et al. (Chem Pharm Bull (Tokyo) 1996 August;44(8):1454-1464)). β-escin is also known as aescin.

Another preferred haemolytic saponin for use in the present invention is Digitonin. Digitonin is described in the Merck index (12.sup.th Edition, entry 3204) as a saponin, being derived from the seeds of Digitalis purpurea and purified according to the procedure described Gisvold et al., J. Am. Pharm. Assoc., 1934, 23, 664; and Ruhenstroth-Bauer, Physiol. Chem., 1955, 301, 621. Its use is described as being a clinical reagent for cholesterol determination.

Another interesting (and thus, preferred) possibility of achieving adjuvant effect is to employ the technique described in Gosselin et al., 1992. In brief, the presentation of a relevant antigen such as an antigen of the present invention can be enhanced by conjugating the antigen to antibodies (or antigen binding antibody fragments) against the F_{C} receptors on monocytes/macrophages. Especially conjugates between antigen and anti-F_{C}RI have been demonstrated to enhance immunogenicity for the purposes of vaccination. The antibody may be conjugated to the respiratory syncytial virus F polypeptide VLP after generation or as a part of the generation including by expressing as a fusion to any one of the polypeptide components of the respiratory syncytial virus F polypeptide VLP.

Other possibilities involve the use of the targeting and immune modulating substances (i.e. cytokines). In addition, synthetic inducers of cytokines such as poly I:C may also be used.

Suitable mycobacterial derivatives may be selected from the group consisting of muramyl dipeptide, complete Freund's adjuvant, RIBI, (Ribi ImmunoChem Research Inc., Hamilton, Mont.) and a diester of trehalose such as TDM and TDE.

Examples of suitable immune targeting adjuvants include CD40 ligand and CD40 antibodies or specifically binding fragments thereof (cf. the discussion above), mannose, a Fab fragment, and CTLA-4.

Examples of suitable polymer adjuvants include a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12-15, 1998, Seascape Resort, Aptos, Calif.”

Oligonucleotides may be used as adjuvants in conjunction with the respiratory syncytial virus F polypeptide VLP vaccines and preferably contain two or more dinucleotide CpG motifs separated by at least three or more preferably at least six or more nucleotides. CpG-containing oligonucleotides (in which the CpG dinucleotide is unmethylated) induce a predominantly Th1 response. Such oligonucleotides are well known and are described, for example, in WO 96/02555, WO 99/33488 and U.S. Pat. Nos. 6,008,200 and 5,856,462.

Such oligonucleotide adjuvants may be deoxynucleotides. In a preferred embodiment the nucleotide backbone in the oligonucleotide is phosphorodithioate, or more preferably a phosphorothioate bond, although phosphodiester and other nucleotide backbones such as PNA are within the scope of the invention including oligonucleotides with mixed backbone linkages. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified nucleotide backbones.

(SEQ ID NO: 1)
OLIGO 1: TCC ATG ACG TTC CTG ACG TT (CpG 1826)
(SEQ ID NO: 2)
OLIGO 2: TCT CCC AGC GTG CGC CAT (CpG 1758)
(SEQ ID NO: 3)
OLIGO 3: ACC GAT GAC GTC GCC GGT GAC GGC
ACC ACG
(SEQ ID NO: 4)
OLIGO 4: TCG TCG TTT TGT COT TTT GTC
GTT (CpG 2006)
(SEQ ID NO: 5)
OLIGO 5: TCC ATG ACG TTC CTG ATG CT (CpG 1668)

Alternative preferred CpG oligonucleotides include the above sequences with inconsequential deletions or additions thereto. The CpG oligonucleotides as adjuvants may be synthesized by any method known in the art (e.g., EP 468520). Preferably, such oligonucleotides may be synthesized utilizing an automated synthesizer. Such oligonucleotide adjuvants may be between 10-50 bases in length. Another adjuvant system involves the combination of a CpG-containing oligonucleotide and a saponin derivative particularly the combination of CpG and QS21 is disclosed in WO 00/09159.

Many single or multiphase emulsion systems have been described. One of skill in the art may readily adapt such emulsion systems for use with respiratory syncytial virus F polypeptide VLPs so that the emulsion does not disrupt the respiratory syncytial virus F polypeptide VLP's structure. Oil in water emulsion adjuvants per se have been suggested to be useful as adjuvant compositions (EPO 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B).

The oil emulsion adjuvants for use with the respiratory syncytial virus F polypeptide VLP vaccines described herein may be natural or synthetic, and may be mineral or organic. Examples of mineral and organic oils will be readily apparent to the man skilled in the art.

In order for any oil in water composition to be suitable for human administration, the oil phase of the emulsion system preferably includes a metabolizable oil. The meaning of the term metabolizable oil is well known in the art. Metabolizable can be defined as “being capable of being transformed by metabolism” (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils are also part of this invention and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolizable oil virtue of the fact that it is an intermediate in the biosynthesis of cholesterol (Merck index, 10th Edition, entry no.8619).

Particularly preferred oil emulsions are oil in water emulsions, and in particular squalene in water emulsions.

In addition, the most preferred oil emulsion adjuvants of the present invention include an antioxidant, which is preferably the oil a-tocopherol (vitamin E, EP 0 382 271 B1).

WO 95/17210 and WO 99/11241 disclose emulsion adjuvants based on squalene, a-tocopherol, and TWEEN 80™, optionally formulated with the immunostimulants QS21 and/or 3D-MPL. WO 99/12565 discloses an improvement to these squalene emulsions with the addition of a sterol into the oil phase. Additionally, a triglyceride, such as tricaprylin (C27H50O6), may be added to the oil phase in order to stabilize the emulsion (WO 98/56414).

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges. The amounts of the components present in the oil emulsions of the present invention are conventionally in the range of from 2 to 10% oil, such as squalene; and when present, from 2 to 10% alpha tocopherol; and from 0.3 to 3% surfactant, such as polyoxyethylene sorbitan monooleate. Preferably the ratio of oil: alpha tocopherol is equal or less than 1 as this provides a more stable emulsion. SPAN 85™ may also be present at a level of about 1%. In some cases it may be advantageous that the respiratory syncytial virus F polypeptide VLP vaccines disclosed herein will further contain a stabilizer.

The method of producing oil in water emulsions is well known to the man skilled in the art. Commonly, the method includes the step of mixing the oil phase with a surfactant such as a PBS/TWEEN80® solution, followed by homogenization using a homogenizer, it would be clear to a man skilled in the art that a method comprising passing the mixture twice through a syringe needle would be suitable for homogenizing small volumes of liquid. Equally, the emulsification process in microfluidizer (M110S microfluidics machine, maximum of 50 passes, for a period of 2 minutes at maximum pressure input of 6 bar (output pressure of about 850 bar)) could be adapted by the man skilled in the art to produce smaller or larger volumes of emulsion. This adaptation could be achieved by routine experimentation comprising the measurement of the resultant emulsion until a preparation was achieved with oil droplets of the required diameter.

The respiratory syncytial virus F polypeptide VLP vaccine preparations disclosed herein may be used to protect or treat a mammal or bird susceptible to, or suffering from a viral infection, by means of administering the vaccine by intranasal, intramuscular, intraperitoneal, intradermal, transdermal, intravenous, or subcutaneous administration. Methods of systemic administration of the vaccine preparations may include conventional syringes and needles, or devices designed for ballistic delivery of solid vaccines (WO 99/27961), or needleless pressure liquid jet device (U.S. Pat. No. 4,596,556; U.S. Pat. No. 5,993,412), or transdermal patches (WO 97/48440; WO 98/28037). The respiratory syncytial virus F polypeptide VLP vaccines may also be applied to the skin (transdermal or transcutaneous delivery WO 98/20734; WO 98/28037). The respiratory syncytial virus F polypeptide VLP vaccines disclosed herein therefore includes a delivery device for systemic administration, pre-filled with the respiratory syncytial virus F polypeptide VLP vaccine or adjuvant compositions. Accordingly there is provided a method for inducing an immune response in an individual preferably mammal or bird, comprising the administration of a vaccine comprising any of the respiratory syncytial virus F polypeptide VLP compositions described herein and optionally including an adjuvant and/or a carrier, to the individual, wherein the vaccine is administered via the parenteral or systemic route.

Preferably the vaccine preparations of the present invention may be used to protect or treat a mammal or bird susceptible to, or suffering from a viral infection, by means of administering the vaccine via a mucosal route, such as the oral/alimentary or nasal route. Alternative mucosal routes are intravaginal and intra-rectal. The preferred mucosal route of administration is via the nasal route, termed intranasal vaccination. Methods of intranasal vaccination are well known in the art, including the administration of a droplet, spray, or dry powdered form of the vaccine into the nasopharynx of the individual to be immunized. Nebulized or aerosolized vaccine formulations are therefore preferred forms of the respiratory syncytial virus F polypeptide VLP vaccines disclosed herein. Enteric formulations such as gastro resistant capsules and granules for oral administration, suppositories for rectal or vaginal administration are also formulations of the respiratory syncytial virus F polypeptide VLP vaccines disclosed herein.

The preferred respiratory syncytial virus F polypeptide VLP vaccine compositions disclosed herein, represent a class of mucosal vaccines suitable for application in humans to replace systemic vaccination by mucosal vaccination.

The respiratory syncytial virus F polypeptide VLP vaccines may also be administered via the oral route. In such cases the pharmaceutically acceptable excipient may also include alkaline buffers, or enteric capsules or microgranules. The respiratory syncytial virus F polypeptide VLP vaccines may also be administered by the vaginal route. In such cases, the pharmaceutically acceptable excipients may also include emulsifiers, polymers such as CARBOPOL®, and other known stabilizers of vaginal creams and suppositories. The respiratory syncytial virus F polypeptide VLP vaccines may also be administered by the rectal route. In such cases the excipients may also include waxes and polymers known in the art for forming rectal suppositories.

Alternatively the respiratory syncytial virus F polypeptide VLP vaccines formulations may be combined with vaccine vehicles composed of chitosan (as described above) or other polycationic polymers, polylactide and polylactide-coglycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM.

Additional illustrative adjuvants for use in the pharmaceutical and vaccine compositions using the respiratory syncytial virus F polypeptide VLPs as described herein include SAF (Chiron, Calif., United States), MF-59 (Chiron, see, e.g., Granoff et al. (1997) Infect Immun. 65 (5):1710-1715), the SBAS series of adjuvants (e.g., SB-AS2 (SmithKline Beecham adjuvant system #2; an oil-in-water emulsion containing MPL and QS21); SBAS-4 (SmithKline Beecham adjuvant system #4; contains alum and MPL), available from SmithKline Beecham, Rixensart, Belgium), Detox (Enhanzyn®) (GlaxoSmithKline), RC-512, RC-522, RC-527, RC-529, RC-544, and RC-560 (GlaxoSmithKline) and other aminoalkyl glucosaminide 4-phosphates (AGPs), such as those described in pending U.S. patent application Ser. Nos. 08/853,826 and 09/074,720.

Other examples of adjuvants include, but are not limited to, Hunter's TiterMax® adjuvants (CytRx Corp., Norcross, Ga.); Gerbu adjuvants (Gerbu Biotechnik GmbH, Gaiberg, Germany); nitrocellulose (Nilsson and Larsson (1992) Res. Immunol. 143:553-557); alum (e.g., aluminum hydroxide, aluminum phosphate) emulsion based formulations including mineral oil, non-mineral oil, water-in-oil or oil-in-water emulsions, such as the Seppic ISA series of Montamide adjuvants (e.g., ISA-51, ISA-57, ISA-720, ISA-151, etc.; Seppic, Paris, France); and PROVAX® (IDEC Pharmaceuticals); OM-174 (a glucosamine disaccharide related to lipid A); Leishmania elongation factor; non-ionic block copolymers that form micelles such as CRL 1005; and Syntex Adjuvant Formulation. See, e.g., O'Hagan et al. (2001) Biomol Eng. 18(3):69-85; and “Vaccine Adjuvants: Preparation Methods and Research Protocols” D. O'Hagan, ed. (2000) Humana Press.

Other preferred adjuvants include adjuvant molecules of the general formula

HO(CH₂CH₂O)ₙ-A-R, (I)

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C.sub.1-50, preferably C.sub.4-C.sub.20 alkyl and most preferably C.sub.12 alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12.sup.th edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described above.

Further examples of suitable pharmaceutically acceptable excipients for use with the respiratory syncytial virus F polypeptide VLP vaccines disclosed herein include water, phosphate buffered saline, isotonic buffer solutions.

This invention will be better understood by reference to the following non-limiting Examples. As described herein, the invention includes respiratory syncytial virus F polypeptide VLPs which optionally comprises an additional lipid raft-associated polypeptide linked to an antigen or adjuvant. The following Examples describe a representative embodiment of the invention which includes respiratory syncytial virus F polypeptide VLPs.

### EXAMPLE 1

This example demonstrates that mammalian cell expression of a truncated version of the RSV fusion (F) glycoprotein lacking the cytoplasmic tail region or hybrid F proteins containing transmembrane and cytoplasmic tail regions derived from influenza hemagglutinin results in the formation of enveloped virus-like particles (VLPs) containing RSV F.

This example was designed such that the RSV F glycoprotein was expressed in mammalian cells with and without co-expression of the murine leukemia virus Gag gene product. The expectation was that the RSV F would be incorporated into enveloped Gag VLPs budding from the cells.

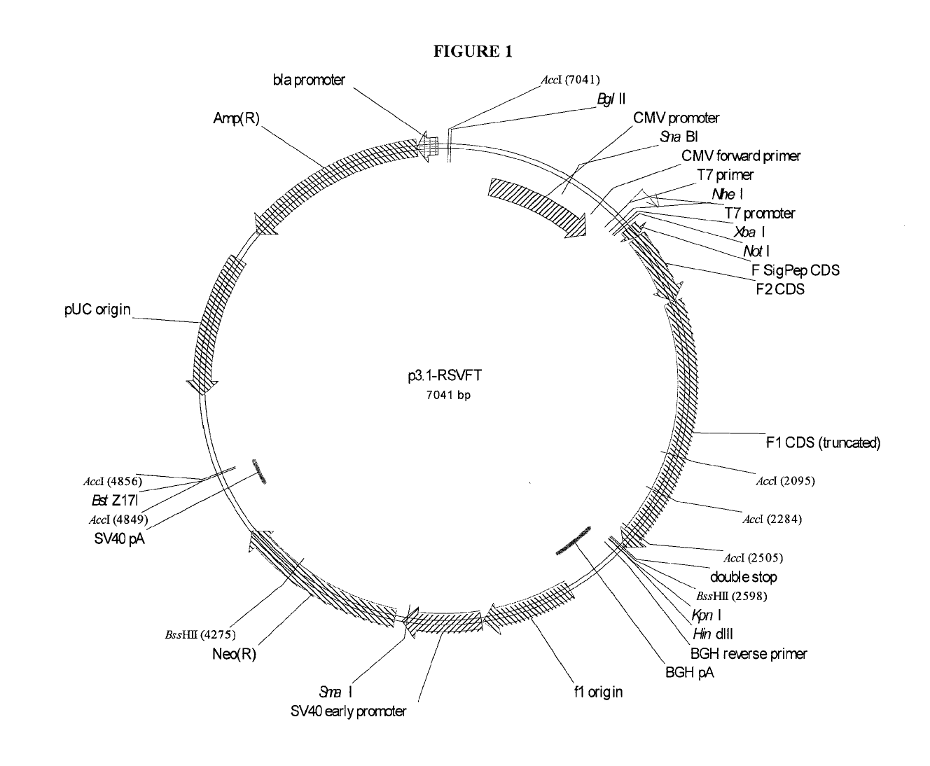

Plasmid p3.1-RSVFT encoded a truncated version of F (RSVFT) devoid of its cytoplasmic tail but containing the native transmembrane domain. A map of p3.1-RSVFT is shown in FIG. 1 and the coding sequence for RSVFT as follows:

(SEQ ID NO: 6)
atggaactgctgatcctgaaggctaacgctatcaccaccatcctgaccgc
tgtcaccttctgcttcgcctccggccagaacatcaccgaggaattctacc
agtccacctgctccgctgtctccaagggttacctgtccgctctgcgcacc
ggctggtacacctccgtcatcaccatcgagctgtccaacatcaaggaaaa
caagtgcaacggcaccgacgctaaggtcaagctgatcaagcaggaactgg
acaagtacaagaacgctgtcaccgagctgcagctgctgatgcagtccacc
cccgctaccaacaaccgcgctcgccgtgagctgccccgcttcatgaacta
caccctgaacaacgccaagaaaaccaacgtcaccctgtccaagaagcgca
agcgccgcttcctgggtttcctgctgggtgtcggttccgctatcgcttcc
ggtgtcgctgtctctaaggtcctgcacctggaaggcgaggtcaacaagat
caagtccgccctgctgtccaccaacaaggctgtcgtgtccctgtccaacg
gtgtctccgtcctgacctccaaggtgctggacctgaagaactacatcgac
aagcagctgctgcccatcgtcaacaagcagtcctgctccatctccaacat
cgagactgtcatcgagttccagcagaagaacaaccgcctgctggaaatca
cccgcgagttctccgtcaacgctggtgtcaccacccctgtctccacctac
atgctgaccaactccgagctgctgtccctgatcaacgacatgcccatcac
caacgaccaaaagaaactgatgtccaacaacgtccagatcgtccgccagc
agtcctactctatcatgagcatcatcaaggaagaggtcctggcttacgtc
gtccagctgcccctgtacggtgtcatcgacaccccctgctggaagctgca
cacctcccccctgtgcaccaccaacaccaaggaaggttccaacatctgcc
tgacccgcaccgaccgcggctggttctgcgacaacgctggctctgtctcc
ttcttcccccaagctgagacttgcaaggtccagtccaaccgcgtgttctg
cgacaccatgaactccctgaccctgccctccgaggtcaacctgtgcaacg
tcgacatcttcaaccccaagtacgactgcaagatcatgacctctaagacc
gacgtgtcctcctctgtcatcacctccctgggtgctatcgtgtcctgcta
cggcaagaccaagtgcaccgcttccaacaagaaccgcggtatcatcaaga
ccttctccaacggttgcgactacgtgtccaacaagggcgtcgacaccgtg
tccgtcggcaacaccctgtactacgtgaacaagcaggaaggcaagtccct
gtacgtcaagggcgagcccatcatcaacttctacgaccccctggtgttcc
cctccgacgagttcgacgcttccatcagccaggtcaacgagaagatcaac
cagtccctggctttcatccgcaagtccgacgagctgctgcacaacgtgaa
cgctggcaagtctaccaccaacatcatgatcaccactatcatcatcgtga
tcatcgtcatcctgctgtctctgatcgctgtcggtctgctgctgtactaa

The RSVFT coding sequence was synthesized as a custom synthetic DNA fragment and was not cloned from virus since the natural RSV F gene is from a paramyxovirus which replicates in the cytoplasm of cells and would therefore not be expected to be appropriately expression in the nucleus of a cell. The encoded amino acid sequence of RSVFT is as follows (with the signal peptide and transmembrane domains in capital letters):

(SEQ ID NO: 7)
MELLILKANAITTILTAVTFCfasgqniteefyqstcsavskgylsalrt
gwytsvitielsnikenkcngtdakvklikqeldkyknavtelqllmqst
patnnrarrelprfmnytlnnakktnvtlskkrkrrflgfllgvgsaias
gvavskvlhlegevnkiksallstnkavvslsngvsvltskvldlknyid
kqllpivnkqscsisnietviefqqknnrlleitrefsvnagvttpvsty
mltnsellslindmpitndqkklmsnnvqivrqqsysimsiikeevlayv
vqlplygvidtpcwklhtsplcttntkegsnicltrtdrgwfcdnagsvs
ffpqaetckvqsnrvfcdtmnsltlpsevnlcnvdifnpkydckimtskt
dvsssvitslgaivscygktkctasnknrgiiktfsngcdyvsnkgvdtv
svgntlyyvnkqegkslyvkgepiinfydplvfpsdefdasisqvnekin
qslafirksdellhnvnagksttnIMITTIIIVIIVILLSLIAVGLLLY

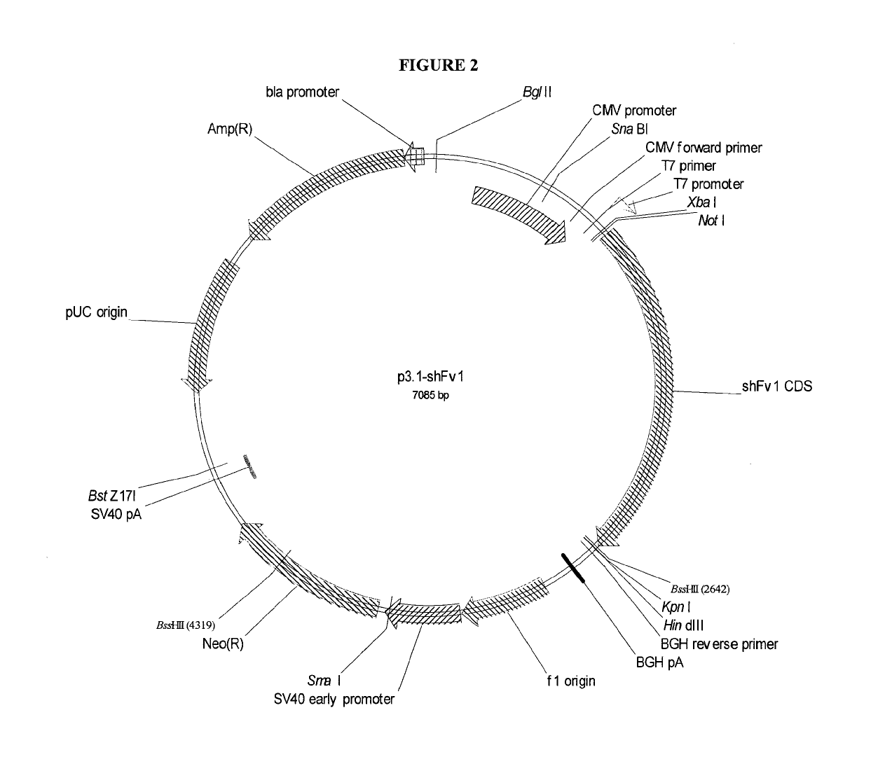

Plasmid p3.1-shFv1 encoded a hybrid protein consisting of the ectodomain of RSV F fused to the transmembrane domain and cytoplasmic tail of an H5 hemagglutinin from influenza A. FIG. 2 shows a map of p3.1-shFv1 and the coding sequence for shFv1 is as follows:

(SEQ ID NO: 8)
atggaactgctgatcctgaaggctaacgctatcaccaccatcctgaccgc
tgtgaccttctgcttcgcttccggccagaacatcaccgaggaattctacc
agtccacctgctccgctgtgtccaagggttacctgtccgctctgcgtacc
ggttggtacacctccgtgatcaccatcgagctgtccaacatcaaagagaa
caagtgcaacggcaccgacgctaaggtcaagctgatcaagcaggaactgg
acaagtacaagaacgctgtgaccgagctgcagctgctgatgcagtccacc
cccgctaccaacaaccgtgctcgtcgtgagctgccccgtttcatgaacta
caccctgaacaacgccaagaaaaccaacgtcaccctgtccaagaagcgta
agcgtcgtttcctgggtttcctgctgggtgtgggtagcgctatcgcctcc
ggtgtcgctgtctccaaggtgctgcacctcgagggcgaggtgaacaagat
caagtccgccctgctgtccaccaacaaggctgtggtgtccctgtccaacg
gtgtctccgttctgaccagcaaggtcttggacctgaagaactacatcgac
aagcagctgctgcccatcgtgaacaagcagtcctgctccatctccaacat
cgagactgtgatcgagttccagcagaagaacaaccgtctgctcgagatca
cccgtgagttctccgtgaacgctggtgtcaccacccccgtgtccacctac
atgctgaccaactccgagctgctgtccctgatcaacgacatgcccatcac
caacgaccagaaaaagctgatgtccaacaacgtgcagatcgtgcgtcagc
agtcctactctatcatgagcatcatcaaagaggaagtcctggcttacgtg
gtgcagctgcccctgtacggtgtcatcgacaccccctgctggaagctgca
cacctcccccctgtgcaccaccaacaccaaagagggttccaacatctgcc
tgacccgtaccgatcgtggttggttctgtgacaacgctggttccgtgtcc
ttcttcccccaagctgagacttgcaaggtgcagtccaaccgtgtgttctg
cgacaccatgaactccctgaccctgccctccgaggtgaacctgtgcaacg
tggacatcttcaaccccaagtacgactgcaagatcatgacctctaagacc
gacgtgtcctcctccgtcatcacctccctgggtgctatcgtgtcctgcta
cggcaagaccaagtgcaccgcttccaacaagaaccgcggtatcatcaaga
ccttctccaacggttgcgactacgtgtccaacaagggtgtcgataccgtg
tccgtcggtaacaccctgtactacgtcaacaagcaggaaggcaagtctct
gtacgtgaagggcgagcccatcatcaacttctacgaccccctggtgttcc
cctccgacgagttcgacgcttccatcagccaggtcaacgagaagatcaac
cagtccctggctttcatccgtaagtccgacgagctgctgcacaacgtcaa
cgctggcaagtccaccaccaacatcctgtccatctactccaccgtggctt
cctccctggctctggctatcatgatggctggtctgtccctgtggatgtgc
tccaacggctccctgcagtgccgtatctgcatctaataa

The amino acid sequence of shFv1 is as follows (with the signal peptide coding sequence and the HA transmembrane and cytoplasmic tail sequences shown in capital letters):

(SEQ ID NO: 9)
MELLILKANAITTILTAVTFcfasgqniteefyqstcsavskgylsalrt
gwytsvitielsnikenkcngtdakvklikqeldkyknavtelqllmqst
patnnrarrelprfmnytlnnakktnvtlskkrkrrflgfllgvgsaias
gvavskvlhlegevnkiksallstnkavvslsngvsvltskvldlknyid
kqllpivnkqscsisnietviefqqknnrlleitrefsvnagvttpvsty
mltnsellslindmpitndqkklmsnnvqivrqqsysimsiikeevlayv
vqlplygvidtpcwklhtsplcttntkegsnicltrtdrgwfcdnagsvs
ffpqaetckvqsnrvfcdtmnsltlpsevnlcnvdifnpkydckimtskt
dvsssvitslgaivscygktkctasnknrgiiktfsngcdyvsnkgvdtv
svgntlyyvnkqegkslyvkgepiinfydplvfpsdefdasisqvnekin
qslafirksdellhnvnagksttnILSIYSTVASSLALAIMMAGLSLWMC
SNGSLQCRICI

As with RSVFT, the shFv1 coding sequence was generated by DNA synthesis.

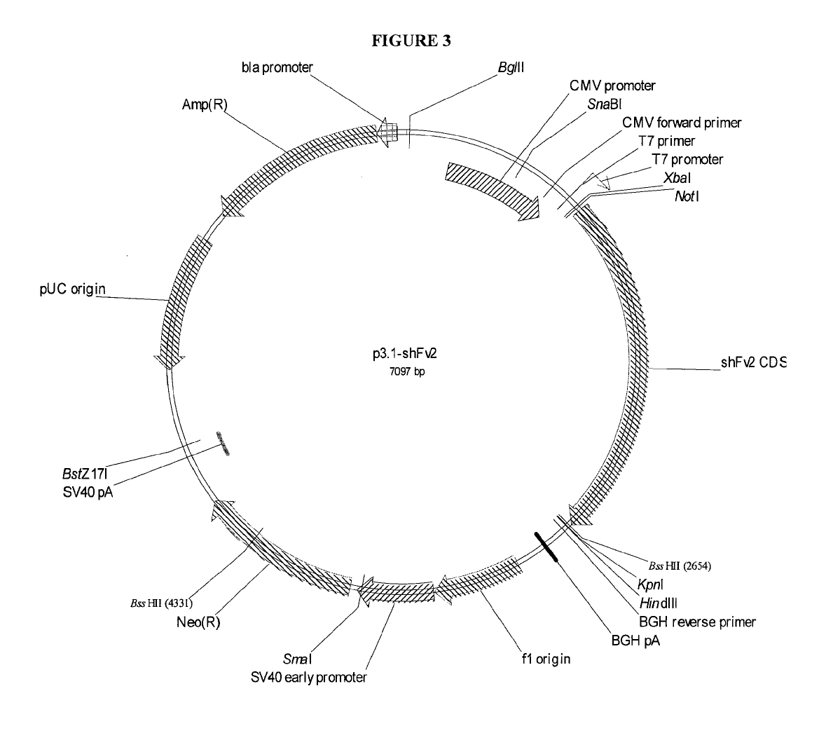

Plasmid p3.1-shFv2 encoded a hybrid protein consisting of the ectodomain of RSV F fused to the transmembrane domain and cytoplasmic tail of an H5 hemagglutinin from influenza A. shFv2 differed from shFv1 in that the influenza HA-derived transmembrane and tail region is four amino acids longer. FIG. 3 shows a plasmid map of p3.1-shFv2. The coding sequence for shFv2 is as follows:

(SEQ ID NO: 10)
atggaactgctgatcctgaaggctaacgctatcaccaccatcctgaccgc
tgtgaccttctgcttcgcttccggccagaacatcaccgaggaattctacc
agtccacctgctccgctgtgtccaagggttacctgtccgctctgcgtacc
ggttggtacacctccgtgatcaccatcgagctgtccaacatcaaagagaa
caagtgcaacggcaccgacgctaaggtcaagctgatcaagcaggaactgg
acaagtacaagaacgctgtgaccgagctgcagctgctgatgcagtccacc
cccgctaccaacaaccgtgctcgtcgtgagctgccccgtttcatgaacta
caccctgaacaacgccaagaaaaccaacgtcaccctgtccaagaagcgta
agcgtcgtttcctgggtttcctgctgggtgtgggtagcgctatcgcctcc
ggtgtcgctgtctccaaggtgctgcacctcgagggcgaggtgaacaagat
caagtccgccctgctgtccaccaacaaggctgtggtgtccctgtccaacg
gtgtctccgttctgaccagcaaggtcttggacctgaagaactacatcgac
aagcagctgctgcccatcgtgaacaagcagtcctgctccatctccaacat
cgagactgtgatcgagttccagcagaagaacaaccgtctgctcgagatca
cccgtgagttctccgtgaacgctggtgtcaccacccccgtgtccacctac
atgctgaccaactccgagctgctgtccctgatcaacgacatgcccatcac
caacgaccagaaaaagctgatgtccaacaacgtgcagatcgtgcgtcagc
agtcctactctatcatgagcatcatcaaagaggaagtcctggcttacgtg
gtgcagctgcccctgtacggtgtcatcgacaccccctgctggaagctgca
cacctcccccctgtgcaccaccaacaccaaagagggttccaacatctgcc
tgacccgtaccgatcgtggttggttctgtgacaacgctggttccgtgtcc
ttcttcccccaagctgagacttgcaaggtgcagtccaaccgtgtgttctg
cgacaccatgaactccctgaccctgccctccgaggtgaacctgtgcaacg
tggacatcttcaaccccaagtacgactgcaagatcatgacctctaagacc
gacgtgtcctcctccgtcatcacctccctgggtgctatcgtgtcctgcta
cggcaagaccaagtgcaccgcttccaacaagaaccgcggtatcatcaaga
ccttctccaacggttgcgactacgtgtccaacaagggtgtcgataccgtg
tccgtcggtaacaccctgtactacgtcaacaagcaggaaggcaagtctct
gtacgtgaagggcgagcccatcatcaacttctacgaccccctggtgttcc
cctccgacgagttcgacgcttccatcagccaggtcaacgagaagatcaac
cagtccctggctttcatccgtaagtccgacgagctgctgcacaacgtcaa
cgctggcaagtccaccaccaacggcacctaccagatcctgtccatctact
ccaccgtggcttcctccctggctctggctatcatgatggctggtctgtcc
ctgtggatgtgctccaacggctccctgcagtgccgtatctgcatctaata
a

The amino acid sequence of shFv2 is as follows (with the signal peptide coding sequence and the HA transmembrane and cytoplasmic tail sequences shown in capital letters):

(SEQ ID NO: 11)
MELLILKANAITTILTAVTFcfasgqniteefyqstcsavskgylsalrt
gwytsvitielsnikenkcngtdakvklikqeldkyknavtelqllmqst
patnnrarrelprfmnytlnnakktnvtlskkrkrrflgfllgvgsaias
gvavskvlhlegevnkiksallstnkavvslsngvsvltskvldlknyid
kqllpivnkqscsisnietviefqqknnrlleitrefsvnagvttpvsty
mltnsellslindmpitndqkklmsnnvqivrqqsysimsiikeevlayv
vqlplygvidtpcwklhtsplcttntkegsnicltrtdrgwfcdnagsvs
ffpqaetckvqsnrvfcdtmnsltlpsevnlcnvdifnpkydckimtskt
dvsssvitslgaivscygktkctasnknrgiiktfsngcdyvsnkgvdtv
svgntlyyvnkqegkslyvkgepiinfydplvfpsdefdasisqvnekin
gslafirksdellhnvnagksttnGTYQILSIYSTVASSLALAIMMAGLS
LWMCSNGSLQCRICI

The shFv2 coding sequence was generated by DNA synthesis.

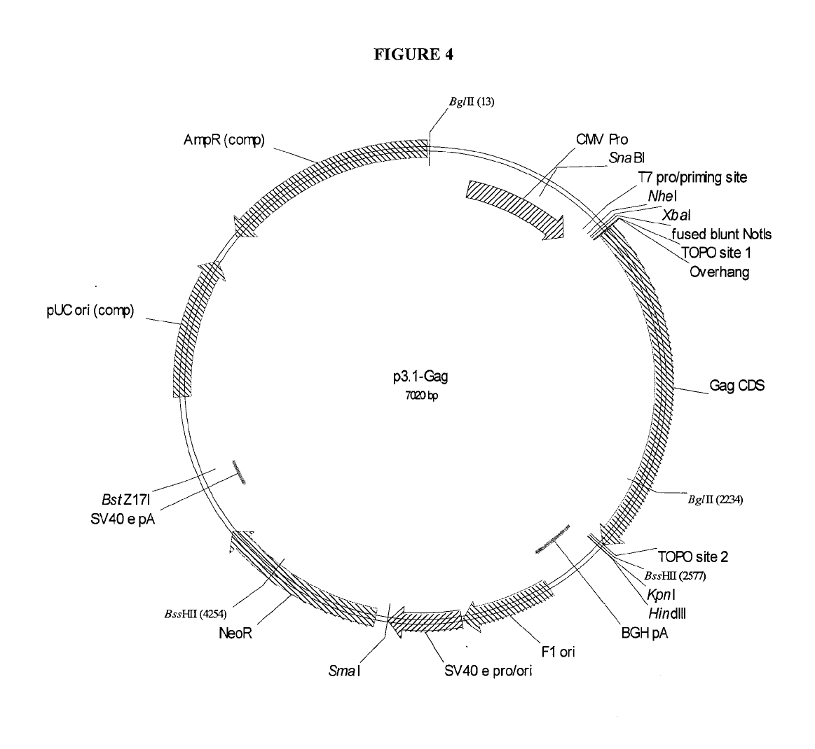

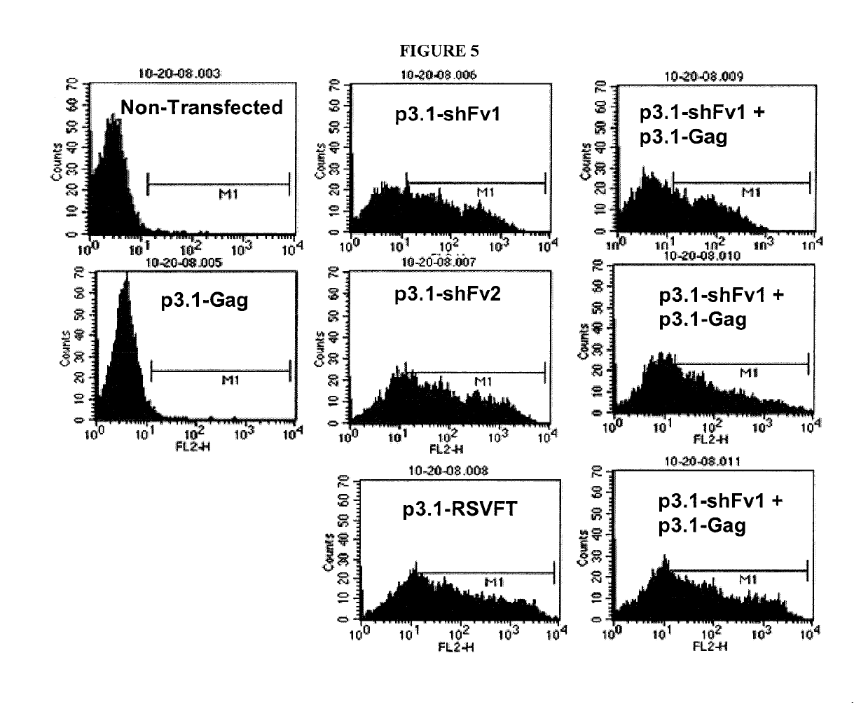

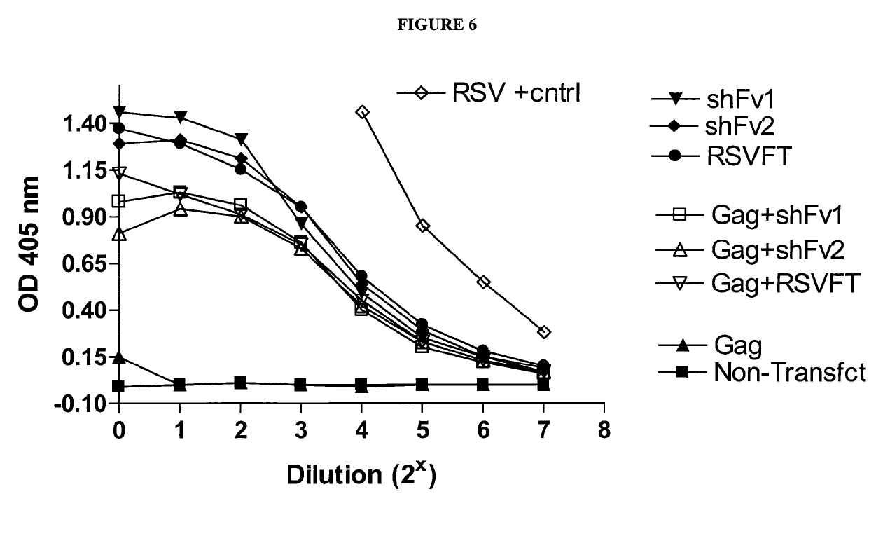

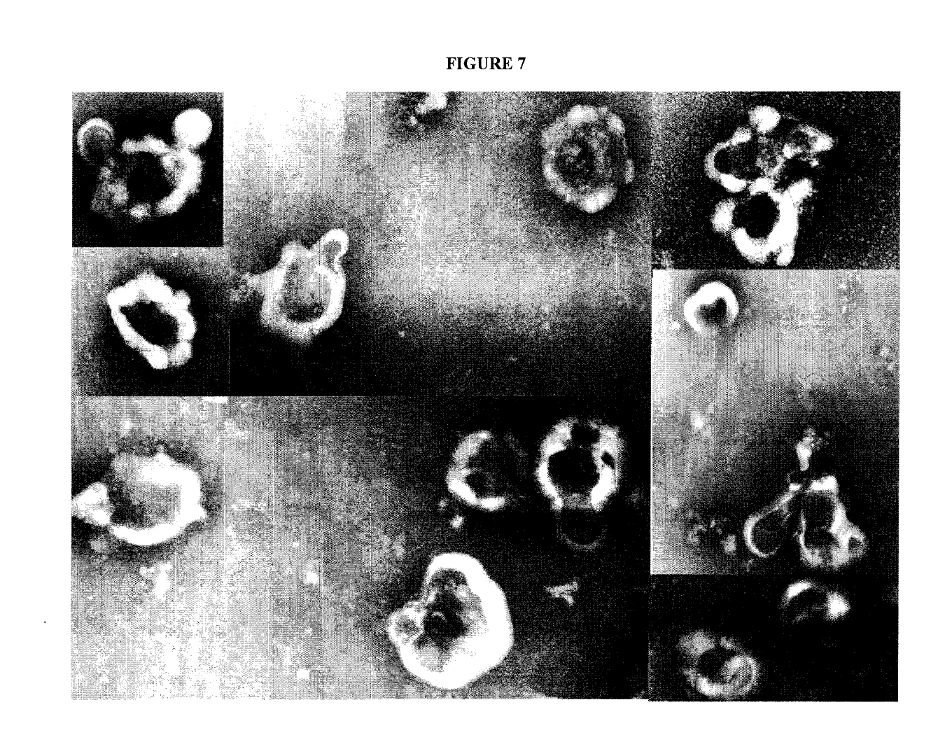

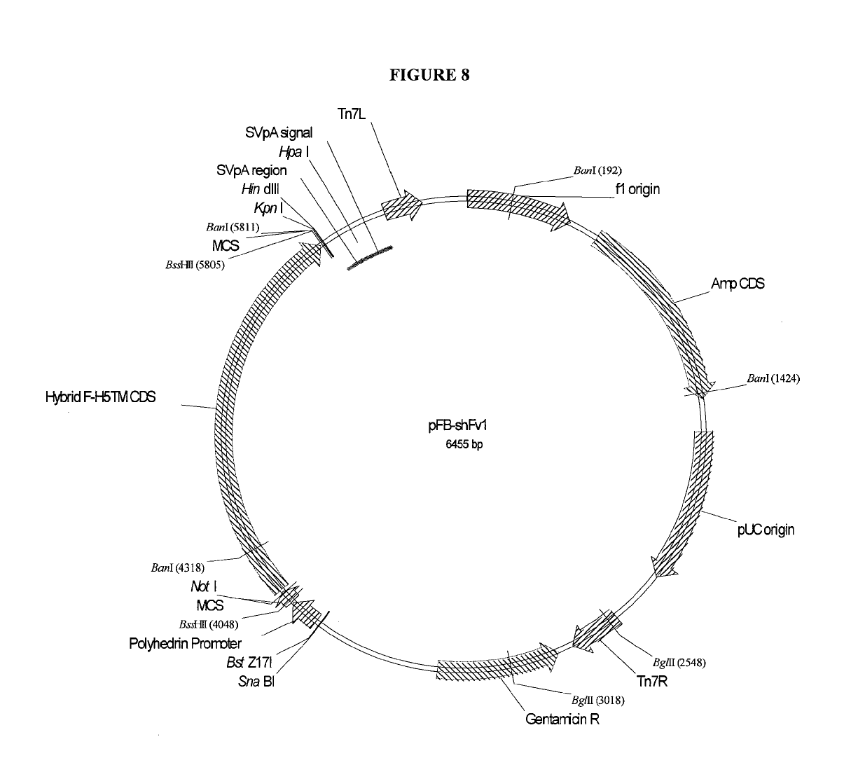

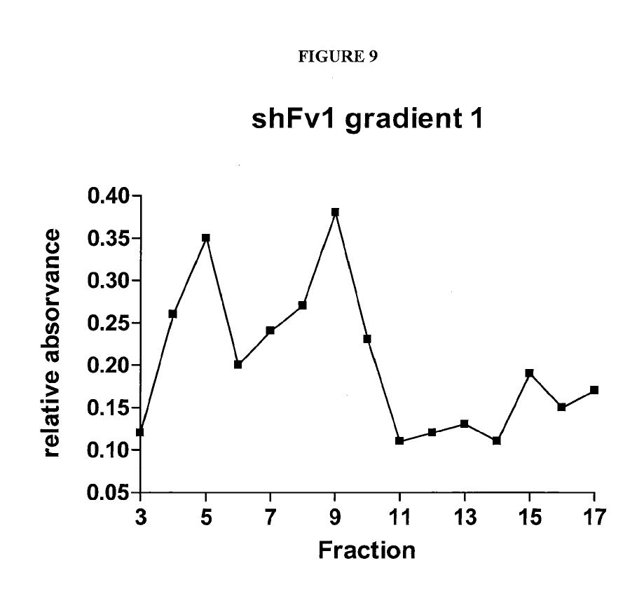

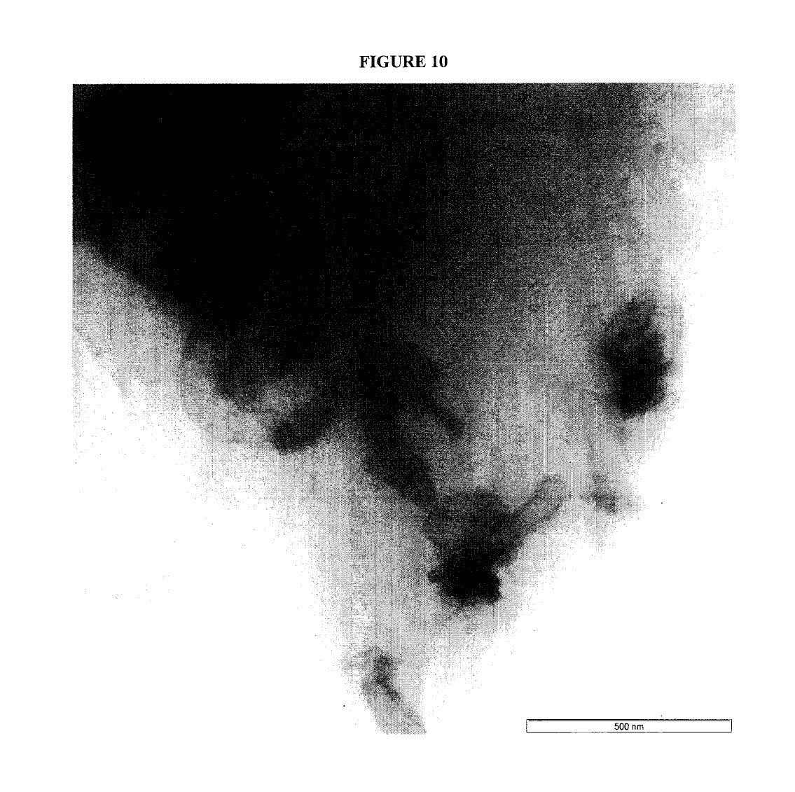

Plasmid p3.1-Gag encoded the Gag gene product from murine leukemia virus. FIG. 4 shows a plasmid map of p3.1 Gag. The coding sequence for Gag from plasmid p3.1-Gag is as follows:

(SEQ ID NO: 12)
atgggccagactgttaccactcccttaagtttgaccttaggtcactggaa
agatgtcgagcggatcgctcacaaccagtcggtagatgtcaagaagagac
gttgggttaccttctgctctgcagaatggccaacctttaacgtcggatgg
ccgcgagacggcacctttaaccgagacctcatcacccaggttaagatcaa
ggtcttttcacctggcccgcatggacacccagaccaggtcccctacatcg
tgacctgggaagccttggcttttgacccccctccctgggtcaagcccttt
gtacaccctaagcctccgcctcctcttcctccatccgccccgtctctccc
ccttgaacctcctcgttcgaccccgcctcgatcctccctttatccagccc
tcactccttctctaggcgccaaacctaaacctcaagttctttctgacagt
ggggggccgctcatcgacctacttacagaagaccccccgccttataggga
cccaagaccacccccttccgacagggacggaaatggtggagaagcgaccc
ctgcgggagaggcaccggacccctccccaatggcatctcgcctacgtggg
agacgggagccccctgtggccgactccactacctcgcaggcattccccct
ccgcgcaggaggaaacggacagcttcaatactggccgttctcctcttctg
acctttacaactggaaaaataataacccttctttttctgaagatccaggt
aaactgacagctctgatcgagtctgttctcatcacccatcagcccacctg
ggacgactgtcagcagctgttggggactctgctgaccggagaagaaaaac
aacgggtgctcttagaggctagaaaggcggtgcggggcgatgatgggcgc
cccactcaactgcccaatgaagtcgatgccgcttttcccctcgagcgccc
agactgggattacaccacccaggcaggtaggaaccacctagtccactatc
gccagttgctcctagcgggtctccaaaacgcgggcagaagccccaccaat
ttggccaaggtaaaaggaataacacaagggcccaatgagtctccctcggc
cttcctagagagacttaaggaagcctatcgcaggtacactccttatgacc
ctgaggacccagggcaagaaactaatgtgtctatgtctttcatttggcag
tctgccccagacattgggagaaagttagagaggttagaagatttaaaaaa
caagacgcttggagatttggttagagaggcagaaaagatctttaataaac
gagaaaccccggaagaaagagaggaacgtatcaggagagaaacagaggaa
aaagaagaacgccgtaggacagaggatgagcagaaagagaaagaaagaga
tcgtaggagacatagagagatgagcaagctattggccactgtcgttagtg
gacagaaacaggatagacagggaggagaacgaaggaggtcccaactcgat
cgcgaccagtgtgcctactgcaaagaaaaggggcactgggctaaagattg
tcccaagaaaccacgaggacctcggggaccaagaccccagacctccctcc
tgaccctagatgactagtag

The amino acid sequence for MLV Gag is as follows:

(SEQ ID NO: 13)
Mgqtvttplsltlghwkdveriahnqsvdvkkrrwvtfcsaewptfnvgw
prdgtfnrdlitqvkikvfspgphghpdqvpyivtwealafdpppwvkpf
vhpkpppplppsapslplepprstpprsslypaltpslgakpkpqvlsds
ggplidlltedpppyrdprpppsdrdgnggeatpageapdpspmasrlrg
rreppvadsttsqafplraggngqlqywpfsssdlynwknnnpsfsedpg
kltaliesvlithqptwddcqqllgtlltgeekqrvllearkavrgddgr
ptqlpnevdaafplerpdwdyttqagrnhlvhyrqlllaglqnagrsptn
lakvkgitqgpnespsaflerlkeayrrytpydpedpgqetnvsmsfiwq
sapdigrklerledlknktlgdlvreaekifnkretpeereerirretee
keerrrtedeqkekerdrrrhremskllatvvsgqkqdrqggerrrsqld
rdqcayckekghwakdcpkkprgprgprpqtslltldd

The Gag gene represented a natural clone from the genome of MLV found in plasmid pAMS (ATCC).

Eight 10 cm² tissue culture dishes were seeded with 293-F cells cultured in Dulbecco's Modified Eagle's medium supplemented with 10% fetal bovine serum. When the cells reached approximately 95% confluence (monolayer culture), each well was transfected with a total of 4 μg of plasmid DNA as follows:
1. No DNA
2. 4 μg p3.1-Gag
3. 4 μg p3.1-RSVFT
4. 4 μg p3.1-shFv1
5. 4 μg p3.1-shFv2
6. 2 μg p3.1-RSVFT+2 μg p3.1-Gag
7. 2 μg p3.1-shFv1+2 μg p3.1-Gag
8. 2 μg p3.1-shFv2+2 μg p3.1-Gag

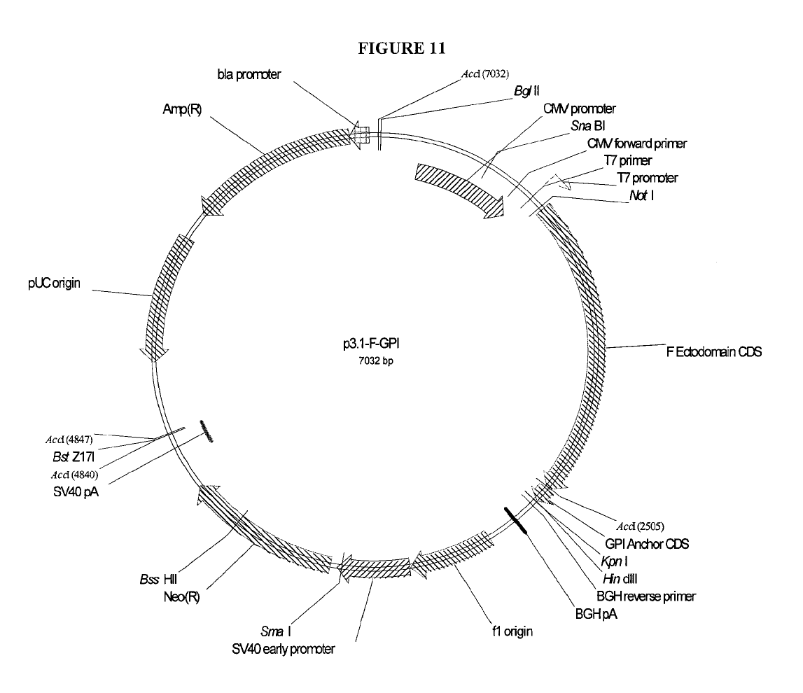

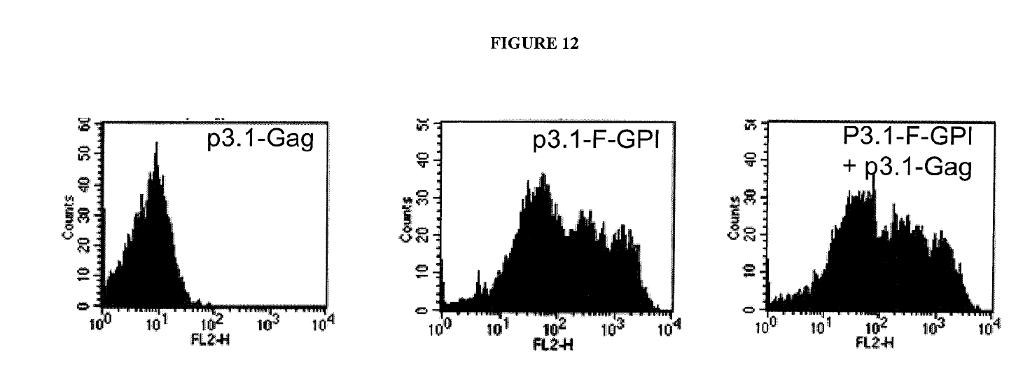

Plasmid DNA was transfected using LIPOFECTAMINE™ 2000 (Invitrogen) according to the manufacture's recommendations. Eight hours post-transfection, the transfection medium was replaced with CD293 serum-free medium and culture was continued. At forty-eight hours post-transfection all dishes containing cells transfected with any of the RSV F expression vectors exhibited significant levels of syncytia (fused cells) consistent with surface expression of correctly folded F. At forty-eight hours post-transfection cells were detached by pipetting and cells and medium were harvested. The expression of RSV F antigenic activity on the surface of cells was further demonstrated by flow cytometry after staining cells with a mouse monoclonal antibody (9C5) that recognizes the correctly folded F antigen in its membrane integrated state. Cells were further reacted with a goat-anti-mouse secondary antibody conjugated to phycoerythrin for fluorescent detection of F-positive cells. FIG. 5 shows the histograms from the flow cytometric runs demonstrating significant levels of surface expression of F on cells transfected with any of the F expression vectors with and without the Gag vector. These data are consistent with the presence of syncytia in transfected cell populations and demonstrate the presence of correctly folded F antigen on the cell surface.

The growth medium harvested from the transfected cells was centrifuged at 2000 rpm for 5 minutes to remove syncytia and any cellular debris and the supernatant from this step was centrifuged over a 30% sucrose cushion in tris-buffered saline at 100,000×g for 1 hour to collect any VLPs that may have been released into the medium as a result of Gag and/or F gene expression. 100,000×g pellets from this step were resuspended in tris-buffered saline for additional analyses.

Resuspended pellets from the ultracentrifuge step were subjected to Western blot analysis to detect the presence of the Gag product as an indication of VLP formation. Unexpectedly, a significant amount of the Gag product was detected in the sample from cells transfected with the p3.1-Gag vector alone, but markedly reduced amounts of Gag were found in pellets from cells transfected with p3.1-Gag in combination with any of the F gene vectors. The results are consistent with expression of the F gene product interfering in some manner with the budding functions of the Gag product.

The presence of the F antigen in resuspended ultracentrifuge pellets was subsequently examined by ELISA as follows: Each resuspended ultracentrifuge pellet sample was loaded into a single well in row A of a Nunc MAXISORP™ flat bottom 96-well ELISA plate and each sample was serially diluted two-fold down each column of the plate. After coating overnight at 4° C., the plate was washed 3 times with PBS containing 0.05% TWEEN 20™ (PBS-T) and then blocked with STARTING BLOCK™ (PBS) (Pierce, Biotechnology). After blocking, 100 μl of 1:1000 diluted monoclonal antibody 9C5 in STARTING BLOCK T20™ (Pierce) was loaded into each well and the plate was incubated at room temperature for 3 hours. The plate was washed again and 100 μl of goat anti-mouse HRP conjugate antibody (Southern Biotech) diluted 1:1000 in STARTING BLOCK T20™ was added to each well and incubated at room temperature for 1.5 hours. Following a final wash (3×) with PBS-T, the plate was developed by the addition of 100 μl ABTS reagent (Pierce) and incubated at room temperature for 45 minutes.

FIG. 6 shows the results of this ELISA in which it can be observed that significant levels of RSV F antigen were detected in the 100,000×g pellets from both F gene alone and F gene+Gag gene transfections. Importantly and unexpectedly, greater amounts of RSV F antigenic activity were observed in the pellets from cells transfected with the RSV F genes alone. These data demonstrates that expression of the F gene alone, in the absence of Gag expression, results in the formation of VLPs carrying the F antigen. This is also consistent with the above observation that co-expression of F and Gag results in a suppression of Gag budding, possibly due to interference of Gag budding by F.

### EXAMPLE 2

This example describes electron microscopic observation of RSV F VLPs released into the medium of mammalian cells transfected with the p3.1-RSVFT vector alone.

To demonstrate that expression of an RSV F gene alone results in the release of VLPs rather than mere aggregates of RSV F, five T75 tissue culture flasks, each containing 293-F cells at approximately 95% confluence in DMEM medium supplemented with 10% FBS were transfected with 30 μg p3.1-RSVFT and 75 μl LIPOFECTAMINE™ 2000 (Invitrogen) according to the manufacture's specifications. At eight hours post-transfection the transfection medium was removed and replaced with CD293 serum-free medium. At forty-eight hours post-transfection significant levels of free-floating syncytia were observed in the media which is consistent with expression of the RSV F gene. At this time the growth medium was harvested and syncytia and cell debris were removed by centrifugation at 2000 rpm for 5 minutes. The medium supernatant was then centrifuged over 30% sucrose cushions in tris-buffered saline at 100,000×g for 1 hour at 10° C. to collect VLPs. Pellets from this centrifugation step were then resuspended in a total of 300 μl tris-buffered saline. The resuspended pellet was then centrifuged briefly at low speed (approximately 5000 rpm) to remove any irreversible protein precipitates or aggregates that formed as a result of ultracentrifugation. The resuspended VLP preparation was then examined by transmission electron microscopy using uranyl acetate negative staining and representative micrographs are shown in FIG. 7. The micrograph collection showed a significant array of enveloped or vesicular particles that exhibit variation in both shapes and sizes. A pleomorphic particle population such as this is consistent with the formation of budded particles that lack a proteinaceous core.

### EXAMPLE 3

This example describes expression of RSV F VLPs in insect cells using the baculovirus expression system which resulted in RSV F VLPs that are highly aggregated with baculovirus particles.

In an attempt to produce RSV F VLPs in the insect cell baculovirus expression system as an alternative to the use of a mammalian cells expression system as described in Example 2, a DNA fragment containing the coding sequence for shFv1 was cloned into the plasmid pFastbacl resulting in the plasmid pFB-shFv1. The map of pFB-shFv1 is shown in FIG. 8. The coding sequence and encoded amino acid sequence for the shFv1 gene are described as above. The plasmid pFB-shFv1 was recombined into the baculovirus genome by transformation into DH10Bac competent cells containing the baculovirus genome in a high molecular weight bacmid. Following selection of transformed clones, high molecular weight bacmid DNA was prepared from these bacterial clones and used for transfection of Sf9 insect cells to generate recombinant baculoviruses containing the shFv1 gene. Details of the procedures involved in producing recombinant baculoviruses using this system can be found in the PDF document at the website tools.invitrogen.com under the file /content/sfs/manuals/bactobac_man.pdf

To produce RSV F VLPs, three 200 ml spinner cultures of Sf9 cells growing in SF900-II medium were incubated at 27° C. until cell densities reached 2 x 10⁶ cells per ml. At that point 20 ml of a passage 2 shFv1-recombinant baculovirus preparation was added to each spinner flask and cultures were incubated at 27° C. until cell viability dropped to below 20%. Culture fluids were then harvested and were clarified of cells and debris by centrifugation at 2k rpm for 15 minutes. The clarified medium was divided into 32 ml aliquots and centrifuged at 100,000×g for 1 hour at 10° C. over 4 ml 30% sucrose cushions containing tris-buffered saline (TBS), pH 7.4, and 0.05% TWEEN 20™. Pellets were resuspended in a total of 12 ml of TBS containing 0.05% TWEEN 20™ and this material was divided into two 6 ml aliquots and centrifuged over 20-60% sucrose density gradients in TB S/0.05% TWEEN 20™. Gradients were fractionated from the bottom. RSV F antigenic activity was detected in individual gradient fractions by the ELISA method described in Example 1 using the 9C5 monoclonal antibody with the following modification: Since high concentrations of sucrose inhibit the ELISA assay, a 100 μl sample of each 1.5 ml gradient fraction was diluted 10-fold with TBS and centrifuged at 100,000×g for 40 minutes to pellet any VLPs that were present out of the sucrose solution. These samples were resuspended in TBS and used directly in the ELISA assay described in Example 1. Results of the ELISA assay are plotted in FIG. 9 showing two prominent peaks of RSV F antigenic activity that coincided with two visible, translucent bands in fractions 5 and 9, respectively. Since aggregated VLPs typically band at higher densities (lower factions) it was anticipated that the band in fraction 9 would contain non-aggregated VLPs. However, electron microscopic examination of material in fraction 9 revealed highly aggregated VLPs and baculovirus particles. A representative micrograph is shown in FIG. 10. It has therefore been determined that mammalian cell expression systems are more appropriate for production and purification of non-aggregated RSV F-containing VLPs.

### EXAMPLE 4

This example demonstrates expression of a GPI-anchored version of RSV F that loses syncytia forming activity but retains VLP budding activity, Gag inhibitory activity, and reactivity with neutralizing monoclonal antibody 9C5.

An attempt was made to create a modified RSV F gene coding sequence that would encode the production of a modified F protein that would target to lipid raft domains but would be devoid of its own VLP budding activity. The purpose of this was to provide an F gene product that would be compatible with the budding activity of MLV Gag such that hybrid Gag-F VLPs could be produced via the ability of the Gag protein to bud from lipid raft domains in which was located the F gene product. To this end the F gene was modified by truncating the transmembrane and cytoplasmic tail coding sequences and replacing them with a sequence encoding a GPI anchor signal. It was expected that the GPI anchor signal would direct the F glycoprotein ectodomain to lipid raft locations but there would be no F polypeptide sequence penetration of the cell membrane at these lipid raft locations. It was expected that this modification would result in the ability of Gag VLPs to bud from these locations forming chimeric Gag-F VLPs. However, surprisingly, the GPI-anchored form of RSV F still demonstrated VLP budding activity demonstrating that any lipid raft-associated polypeptide may be used to direct the RSV F polypeptide to lipid rafts to allow VLP budding by the RSV F polypeptide alone.

FIG. 11 shows a map of the expression vector p3.1-F-GPI in which the chimeric F protein fused to the GPI anchor signal of human carboxypeptidase M was encoded. The nucleotide sequence of the F-GPI coding sequence from plasmid p3.1-F-GPI is a follows:

(SEQ ID NO: 14)
atggagctgctgatcctgaaggccaacgccatcaccaccatcctgaccgc
cgtgaccttctgcttcgcctccggccagaacatcaccgaggagttctacc
agtccacctgctccgccgtgtccaagggctacctgtccgccctgcggacc
ggctggtacacctccgtgatcaccatcgagctgtccaacatcaaagaaaa
caagtgcaacggcaccgacgccaaggtgaagctgatcaagcaggagctgg
acaagtacaagaacgccgtgaccgagctgcagctgctgatgcagtccacc
cctgccaccaacaaccgggccaggcgggagctgcctcggttcatgaacta
caccctgaacaacgccaagaaaaccaacgtcaccctgtccaagaagcgga
agcggcggttcctgggcttcctgctgggcgtgggctccgctatcgcctct
ggcgtggccgtgtctaaggtgctgcacctggagggcgaggtgaacaagat
caagtctgccctgctgtccaccaacaaggccgtggtgtccctgtccaacg
gcgtgtccgtgctgacctccaaggtgctggatctgaagaactacatcgac
aagcagctgctgcctatcgtgaacaagcagtcctgctccatctccaacat
cgagacagtgatcgagttccagcagaagaacaaccggctgctggaaatca
caagagagttctccgtcaacgctggtgtgaccactcctgtctctacttat
atgctgaccaactccgagctgctgtccctgatcaacgacatgcctatcac
caacgaccagaaaaagctgatgtccaacaacgtgcagatcgtgcggcagc
agtcctactctatcatgagcatcatcaaggaggaggtcctggcctacgtg
gtgcagctgcctctgtacggcgtgatcgacaccccttgctggaagctgca
cacctcccccctgtgcaccaccaacaccaaggagggctccaacatctgcc
tgacccggaccgaccggggctggttctgcgacaacgccggctccgtgtcc
ttctttccacaggccgagacatgcaaggtgcagtccaaccgggtgttctg
cgataccatgaactccctgaccctgccttccgaggtgaacctgtgcaacg
tggacatcttcaaccctaagtacgactgcaagatcatgacctctaagacc
gacgtgtcctcctctgtgatcacctccctgggcgccatcgtgtcctgcta
cggcaagaccaagtgcaccgcctccaacaagaaccggggaatcatcaaga
ccttctccaacggctgcgactacgtgtccaataagggcgtggacaccgtg
tccgtgggcaacacactgtactacgtgaataagcaggagggcaagtctct
gtacgtgaagggcgagcctatcatcaacttctacgaccctctggtgttcc
cttccgacgagttcgacgcctccatcagccaggtgaacgagaagatcaac
cagtccctggccttcatccggaagtccgacgagctgctgcacaacgtgaa
cgctggcaagtctaccaccaaccccgaccactccgccgccaccaagccct
ccctgttcctgttcctggtgtccctgctgcacatcttcttcaagtgataa

The amino acid sequence of the F-GPI chimeric protein consisting of the ectodomain of RSV F fused to the GPI anchor signal of human carboxypeptidase M is as follows (with the GPI anchor signal shown in capital letters):

(SEQ ID NO: 15)
mellilkanaittiltavtfcfasgqniteefyqstcsavskgylsalrt
gwytsvitielsnikenkcngtdakvklikqeldkyknavtelqllmqst
patnnrarrelprfmnytlnnakktnvtlskkrkrrflgfllgvgsaias
gvavskvlhlegevnkiksallstnkavvslsngvsvltskvldlknyid
kqllpivnkqscsisnietviefqqknnrlleitrefsvnagvttpvsty
mltnsellslindmpitndqkklmsnnvqivrqqsysimsiikeevlayv
vqlplygvidtpcwklhtsplcttntkegsnicltrtdrgwfcdnagsvs
ffpqaetckvqsnrvfcdtmnsltlpsevnlcnvdifnpkydckimtskt
dvsssvitslgaivscygktkctasnknrgiiktfsngcdyvsnkgvdtv
svgntlyyvnkqegkslyvkgepiinfydplvfpsdefdasisqvnekin
gslafirksdellhnvnagksttnPDHSAATKPSLFLFLVSLLHIFFK

An expression test was conducted in 293F cells in which cells were transfected with the plasmid p3.1-Gag, or p3.1-F-GPI, or a combination of p3.1-Gag and p3.1-F-GPI with the intent of demonstrating the Gag VLPs could be produced that would also carry the RSV F ectodomain.

Twelve 10 cm² tissue culture dishes were seeded with 293-F cells cultured in Dulbecco's Modified Eagle's medium supplemented with 10% fetal bovine serum. When the cells reached approximately 95% confluence (monolayer culture), each well was transfected with a total of 4 μg of plasmid DNA as follows:
Dishes 1-4: p3.1-Gag
Dishes 5-8: p3.1-F-GPI
Dishes 9-12: p3.1-Gag+p3.1-F-GPI

Plasmid DNA was transfected using LIPOFECTAMINE™ 2000 (Invitrogen) according to the manufacture's recommendations. Eight hours post-transfection, the transfection medium was replaced with CD293 serum-free medium and culture was continued. At forty-eight hours post-transfection the dishes containing cells transfected with the p3.1-F-GPI expression vector did not exhibit significant levels of syncytia (fused cells) suggesting that the GPI anchor modification of the F glycoprotein interfered with the ability of the F glycoprotein to promote cell fusion.

At forty-eight hours post-transfection cells were detached by pipetting and cells and medium were harvested. The expression of RSV F antigenic activity on the surface of cells was demonstrated by flow cytometry after staining cells with a mouse monoclonal antibody (9C5) that recognizes the correctly folded F antigen in its membrane integrated state. Cells were further reacted with a goat-anti-mouse secondary antibody conjugated to phycoerythrin for fluorescent detection of F-positive cells. FIG. 12 shows the histograms from the flow cytometric runs demonstrating significant levels of surface expression of F on cells transfected with the p3.1-F-GPI vector with and without the Gag vector. These data demonstrated the presence of correctly folded F antigen on the cell surface even though syncytia forming activity was no longer present.

The growth medium harvested from the transfected cells was centrifuged at 2000 rpm for 5 minutes to remove cells and debris and the supernatant from this step was centrifuged over a 30% sucrose cushion in tris-buffered saline at 100,000×g for 1 hour to collect any VLPs that may have been released into the medium as a result of Gag and/or F-GPI gene expression. 100,000×g pellets from this step were resuspended in tris-buffered saline for additional analyses.

Resuspended pellets from the ultracentrifuge step were subjected to Western blot analysis to detect the presence of the Gag product as an indication of VLP formation. Unexpectedly, a significant amount of the Gag product was detected in the sample from cells transfected with the p3.1-Gag vector alone, but little if any Gag was found in the pellet from cells transfected with p3.1-Gag in combination with the p3.1-F-GPI vector. These results demonstrated that expression of the F-GPI gene product still interferes with the budding functions of the Gag protein even though the mechanism of lipid raft targeting of the chimeric F product has been altered and its transmembrane and cytoplasmic tail regions have been eliminated. These results are shown if FIG. 13 in which Western blots for Gag and RSV F are shown. The RSV Western blot showed that F antigenic activity was found in the pellets from both the F-GPI and the F-GPI+Gag transfections showed that the presence or absence of Gag does not affect F-GPI-mediated budding and particle formation.

The presence of the F antigen in resuspended ultracentrifuge pellets was also examined by ELISA as follows: Each resuspended ultracentrifuge pellet sample was loaded into a single well in row A of a Nunc MAXISORP™ flat bottom 96-well ELISA plate and each sample was serially diluted two-fold down each column of the plate. After coating overnight at 4° C., the plate was washed 3 times with PBS containing 0.05% Tween 20 (PBS-T) and then blocked with STARTING BLOCK™ (PBS) (Pierce, Biotechnology). After blocking, 100 μl of 1:1000 diluted monoclonal antibody 9C5 in STARTING BLOCK™ T20 (Pierce) was loaded into each well and the plate was incubated at room temperature for 3 hours. The plate was washed again and 100 μl of goat anti-mouse HRP conjugate antibody (Southern Biotech) diluted 1:1000 in STARTING BLOCK™ T20 was added to each well and incubated at room temperature for 1.5 hours. Following a final wash (3×) with PBS-T, the plate was developed by the addition of 100 μl ABTS reagent (Pierce) and incubated at room temperature for 45 minutes.

FIG. 14 shows the results of this ELISA in which it can be observed that significant levels of RSV F antigenic activity were detected in the 100,000×g pellets from both F-GPI alone and F-GPI+Gag vector transfected cells Importantly, equal amounts of RSV F antigenic activity were observed in the pellets from cells transfected with the F-GPI and F-GPI+Gag vectors. These data are consistent with the conclusion that expression of the F-GPI gene alone, in the absence of Gag expression, results in the formation of VLPs carrying the F-GPI antigen. This is also consistent with the above observation that co-expression of F-GPI and Gag results in a suppression of Gag budding, possibly due to interference of Gag budding by F-GPI. Thus, modification of the F polypeptide by truncation and addition of the GPI anchor signal does not disrupt the budding activity of F nor does it abrogate the inhibitory effects against Gag budding. This experiment also shows that F-GPI budded particles behave similar to live RSV virions in terms of F antigenic activity detected by the 9C5 monoclonal antibody.

### ADDITIONAL REFERENCES

1. Katz, J. M., W. Lim, C. B. Bridges, T. Rowe, J. Hu-Primmer, X. Lu, R. A. Abernathy, M. Clarke, L. Conn, H. Kwong, M. Lee, G. Au, Y. Y. Ho, K. H. Mak, N. J. Cox, and K. Fukuda. 1999. Antibody response in individuals infected with avian influenza A (H5N1) viruses and detection of anti-H5 antibody among household and social contacts. J Infect Dis 180:1763.

2. Peiris, J. S., W. C. Yu, C. W. Leung, C. Y. Cheung, W. F. Ng, J. M. Nicholls, T. K. Ng, K. H. Chan, S. T. Lai, W. L. Lim, K. Y. Yuen, and Y. Guan. 2004. Re-emergence of fatal human influenza A subtype H5N1 disease. Lancet 363:617.

3. Horimoto, T., N. Fukuda, K. Iwatsuki-Horimoto, Y. Guan, W. Lim, M. Peiris, S. Sugii, T. Odagiri, M. Tashiro, and Y. Kawaoka. 2004. Antigenic differences between H5N1 human influenza viruses isolated in 1997 and 2003. J Vet Med Sci 66:303.

4. Tran, T. H., T. L. Nguyen, T. D. Nguyen, T. S. Luong, P. M. Pham, V. C. Nguyen, T. S. Pham, C. D. Vo, T. Q. Le, T. T. Ngo, B. K. Dao, P. P. Le, T. T. Nguyen, T. L. Hoang, V. T. Cao, T. G. Le, D. T. Nguyen, H. N. Le, K. T. Nguyen, H. S. Le, V. T. Le, D. Christiane, T. T. Tran, J. Menno de, C. Schultsz, P. Cheng, W. Lim, P. Horby, and J. Farrar. 2004. Avian influenza A (H5N1) in 10 patients in Vietnam. N Engl J Med 350:1179.

5. Li, K. S., Y. Guan, J. Wang, G. J. Smith, K. M. Xu, L. Duan, A. P. Rahardjo, P. Puthavathana, C. Buranathai, T. D. Nguyen, A. T. Estoepangestie, A. Chaisingh, P. Auewarakul, H. T. Long, N. T. Hanh, R. J. Webby, L. L. Poon, H. Chen, K. F. Shortridge, K. Y. Yuen, R. G. Webster, and J. S. Peiris. 2004. Genesis of a highly pathogenic and potentially pandemic H5N1 influenza virus in eastern Asia. Nature 430:209.

6. Lipatov, A. S., E. A. Govorkova, R. J. Webby, H. Ozaki, M. Peiris, Y. Guan, L. Poon, and R. G. Webster. 2004. Influenza: emergence and control. J Virol 78:8951.

7. Lipatov, A. S., R. J. Webby, E. A. Govorkova, S. Krauss, and R. G. Webster. 2005. Efficacy of H5 influenza vaccines produced by reverse genetics in a lethal mouse model. J Infect Dis 191:1216.

8. Stephenson, I., K. G. Nicholson, J. M. Wood, M. C. Zambon, and J. M. Katz. 2004. Confronting the avian influenza threat: vaccine development for a potential pandemic. Lancet Infect Dis 4:499.

9. Liu, M., J. M. Wood, T. Ellis, S. Krauss, P. Seiler, C. Johnson, E. Hoffmann, J. Humberd, D. Hulse, Y. Zhang, R. G. Webster, and D. R. Perez. 2003. Preparation of a standardized, efficacious agricultural H5N3 vaccine by reverse genetics. Virology 314:580.

10. Subbarao, K., H. Chen, D. Swayne, L. Mingay, E. Fodor, G. Brownlee, X. Xu, X. Lu, J. Katz, N. Cox, and Y. Matsuoka. 2003. Evaluation of a genetically modified reassortant H5N1 influenza A virus vaccine candidate generated by plasmid-based reverse genetics. Virology 305:192.

11. Webby, R. J., D. R. Perez, J. S. Coleman, Y. Guan, J. H. Knight, E. A. Govorkova, L. R. McClain-Moss, J. S. Peiris, J. E. Rehg, E. I. Tuomanen, and R. G. Webster. 2004. Responsiveness to a pandemic alert: use of reverse genetics for rapid development of influenza vaccines. Lancet 363:1099.

12. Treanor, J. J., B. E. Wilkinson, F. Masseoud, J. Hu-Primmer, R. Battaglia, D. O'Brien, M. Wolff, G. Rabinovich, W. Blackwelder, and J. M. Katz. 2001. Safety and immunogenicity of a recombinant hemagglutinin vaccine for H5 influenza in humans. Vaccine 19:1732.

13. Stephenson, I., R. Bugarini, K. G. Nicholson, A. Podda, J. M. Wood, M. C. Zambon, and J. M. Katz. 2005. Cross-reactivity to highly pathogenic avian influenza H5N1 viruses after vaccination with nonadjuvanted and MF59-adjuvanted influenza A/Duck/Singapore/97 (H5N3) vaccine: a potential priming strategy. J Infect Dis 191:1210.

14. Nicholson, K. G., A. E. Colegate, A. Podda, I. Stephenson, J. Wood, E. Ypma, and M. C. Zambon. 2001. Safety and antigenicity of non-adjuvanted and MF59-adjuvanted influenza A/Duck/Singapore/97 (H5N3) vaccine: a randomised trial of two potential vaccines against H5N1 influenza. Lancet 357:1937.

15. Subbarao, K., B. R. Murphy, and A. S. Fauci. 2006. Development of effective vaccines against pandemic influenza. Immunity 24:5.

16. Kuper, C. F., P. J. Koornstra, D. M. Hameleers, J. Biewenga, B. J. Spit, A. M. Duijvestijn, P. J. van Breda Vriesman, and T. Sminia 1992. The role of nasopharyngeal lymphoid tissue. Immunol Today 13:219.

17. Liang, B., L. Hyland, and S. Hou. 2001. Nasal-associated lymphoid tissue is a site of long-term virus-specific antibody production following respiratory virus infection of mice. J Virol 75:5416.

18. Zuercher, A. W., S. E. Coffin, M. C. Thurnheer, P. Fundova, and J. J. Cebra. 2002. Nasal-associated lymphoid tissue is a mucosal inductive site for virus-specific humoral and cellular immune responses. J Immunol 168:1796.

19. Brandtzaeg, P. 1989. Overview of the mucosal immune system. Curr Top Microbiol Immunol 146:13.

20. Takada, A., S. Matsushita, A. Ninomiya, Y. Kawaoka, and H. Kida. 2003. Intranasal immunization with formalin-inactivated virus vaccine induces a broad spectrum of heterosubtypic immunity against influenza A virus infection in mice. Vaccine 21:3212.

21. Tamura, S. I., H. Asanuma, Y. Ito, Y. Hirabayashi, Y. Suzuki, T. Nagamine, C. Aizawa, T. Kurata, and A. Oya. 1992. Superior cross-protective effect of nasal vaccination to subcutaneous inoculation with influenza hemagglutinin vaccine. Eur J Immunol 22:477.

22. Tumpey, T. M., M. Renshaw, J. D. Clements, and J. M. Katz. 2001. Mucosal delivery of inactivated influenza vaccine induces B-cell-dependent heterosubtypic cross-protection against lethal influenza A H5N1 virus infection. J Virol 75:5141.

23. Kang, S. M., L. Guo, Q. Yao, I. Skountzou, and R. W. Compans. 2004. Intranasal immunization with inactivated influenza virus enhances immune responses to coadministered simian-human immunodeficiency virus-like particle antigens. J Virol 78:9624.

24. Guo, L., X. Lu, S. M. Kang, C. Chen, R. W. Compans, and Q. Yao. 2003. Enhancement of mucosal immune responses by chimeric influenza HA/SHIV virus-like particles. Virology 313:502.

25. Yao, Q., R. Zhang, L. Guo, M. Li, and C. Chen. 2004. Th cell-independent immune responses to chimeric hemagglutinin/simian human immunodeficiency virus-like particles vaccine. J Immunol 173:1951.

26. Latham, T., and J. M. Galarza. 2001. Formation of wild-type and chimeric influenza virus-like particles following simultaneous expression of only four structural proteins. J Virol 75:6154.

27. Galarza, J. M., T. Latham, and A. Cupo. 2005. Virus-like particle vaccine conferred complete protection against a lethal influenza virus challenge. Viral Immunol 18:365.

28. Fromantin, C., B. Jamot, J. Cohen, L. Piroth, P. Pothier, and E. Kohli. 2001. Rotavirus 2/6 virus-like particles administered intranasally in mice, with or without the mucosal adjuvants cholera toxin and Escherichia coli heat-labile toxin, induce a Thl/Th2-like immune response. J Virol 75:11010.

29. Harrington, P. R., B. Yount, R. E. Johnston, N. Davis, C. Moe, and R. S. Baric. 2002. Systemic, mucosal, and heterotypic immune induction in mice inoculated with Venezuelan equine encephalitis replicons expressing Norwalk virus-like particles. J Virol 76:730.

30. Shi, W., J. Liu, Y. Huang, and L. Qiao. 2001. Papillomavirus pseudovirus: a novel vaccine to induce mucosal and systemic cytotoxic T-lymphocyte responses. J Virol 75:10139.

31. Han, M. G., S. Cheetham, M. Azevedo, C. Thomas, and L. J. Saif. 2006 Immune responses to bovine norovirus-like particles with various adjuvants and analysis of protection in gnotobiotic calves. Vaccine 24:317.

32. Illum, L. 1998. Chitosan and its use as a pharmaceutical excipient. Pharm Res 15:1326.

33. Illum, L., I. Jabbal-Gill, M. Hinchcliffe, A. N. Fisher, and S. S. Davis. 2001. Chitosan as a novel nasal delivery system for vaccines. Adv Drug Deliv Rev 51:81.

34. Soane, R. J., M. Hinchcliffe, S. S. Davis, and L. Illum. 2001. Clearance characteristics of chitosan based formulations in the sheep nasal cavity. Int J Pharm 217:183.

35. Baudner, B. C., M. M. Giuliani, J. C. Verhoef, R. Rappuoli, H. E. Junginger, and G. D. Giudice. 2003. The concomitant use of the LTK63 mucosal adjuvant and of chitosan-based delivery system enhances the immunogenicity and efficacy of intranasally administered vaccines. Vaccine 21:3837.

36. Fujihashi, K., T. Koga, F. W. van Ginkel, Y. Hagiwara, and J. R. McGhee. 2002. A dilemma for mucosal vaccination: efficacy versus toxicity using enterotoxin-based adjuvants. Vaccine 20:2431.

37. Mutsch, M., W. Zhou, P. Rhodes, M. Bopp, R. T. Chen, T. Linder, C. Spyr, and R. Steffen. 2004. Use of the inactivated intranasal influenza vaccine and the risk of Bell's palsy in Switzerland. N Engl J Med 350:896.

38. Baldridge, J. R., Y. Yorgensen, J. R. Ward, and J. T. Ulrich. 2000. Monophosphoryl lipid A enhances mucosal and systemic immunity to vaccine antigens following intranasal administration. Vaccine 18:2416.

39. Baldrick, P., D. Richardson, G. Elliott, and A. W. Wheeler. 2002. Safety evaluation of monophosphoryl lipid A (MPL): an immunostimulatory adjuvant. Regul Toxicol Pharmacol 35:398.

40. Baldridge, J. R., P. McGowan, J. T. Evans, C. Cluff, S. Mossman, D. Johnson, and D. Persing. 2004. Taking a Toll on human disease: Toll-like receptor 4 agonists as vaccine adjuvants and monotherapeutic agents. Expert Opin Biol Ther 4:1129.

41. Baldridge, J. GlaxoSmithKline, Personal Communication.

42. Huang, J., R. J. Garmise, T. M. Crowder, K. Mar, C. R. Hwang, A. J. Hickey, J. A. Mikszta, and V. J. Sullivan. 2004. A novel dry powder influenza vaccine and intranasal delivery technology: induction of systemic and mucosal immune responses in rats. Vaccine 23:794.

43. Mills, K. H., C. Cosgrove, E. A. McNeela, A. Sexton, R. Giemza, I. Jabbal-Gill, A. Church, W. Lin, L. Illum, A. Podda, R. Rappuoli, M. Pizza, G. E. Griffin, and D. J. Lewis. 2003. Protective levels of diphtheria-neutralizing antibody induced in healthy volunteers by unilateral priming-boosting intranasal immunization associated with restricted ipsilateral mucosal secretory immunoglobulin a. Infect Immun 71:726.

44. Wimer-Mackin, S., M. Hinchcliffe, C. R. Petrie, S. J. Warwood, W. T. Tino, M. S. Williams, J. P. Stenz, A. Cheff, and C. Richardson. 2006. An intranasal vaccine targeting both the Bacillus anthracis toxin and bacterium provides protection against aerosol spore challenge in rabbits. Vaccine in press.

45. Noad, R., and P. Roy. 2003. Virus-like particles as immunogens. Trends Microbiol 11:438.

46. Yao, Q., V. Vuong, M. Li, and R. W. Compans. 2002. Intranasal immunization with SIV virus-like particles (VLPs) elicits systemic and mucosal immunity. Vaccine 20:2537.

47. Pushko, P., T. M. Tumpey, F. Bu, J. Knell, R. Robinson, and G. Smith. 2005. Influenza virus-like particles comprised of the HA, NA, and M1 proteins of H9N2 influenza virus induce protective immune responses in BALB/c mice. Vaccine 23:5751.

48. Baumert, T. F., S. Ito, D. T. Wong, and T. J. Liang. 1998. Hepatitis C virus structural proteins assemble into viruslike particles in insect cells. J Virol 72:3827.

49. Yao, Q., Z. Bu, A. Vzorov, C. Yang, and R. W. Compans. 2003. Virus-like particle and DNA-based candidate AIDS vaccines. Vaccine 21:638.

50. Tacket, C. O., M. B. Sztein, G. A. Losonsky, S. S. Wasserman, and M. K. Estes. 2003. Humoral, mucosal, and cellular immune responses to oral Norwalk virus-like particles in volunteers. Clin Immunol 108:241.

51. Gomez-Puertas, P., C. Albo, E. Perez-Pastrana, A. Vivo, and A. Portela. 2000. Influenza virus matrix protein is the major driving force in virus budding. J Virol 74:11538.

52. Gheysen, D., E. Jacobs, F. de Foresta, C. Thiriart, M. Francotte, D. Thines, and M. De Wilde. 1989. Assembly and release of HIV-1 precursor Pr55gag virus-like particles from recombinant baculovirus-infected insect cells. Cell 59:103.

53. Johnson, M. C., H. M. Scobie, and V. M. Vogt. 2001. PR domain of rous sarcoma virus Gag causes an assembly/budding defect in insect cells. J Virol 75:4407.

54. Kakker, N. K., M. V. Mikhailov, M. V. Nermut, A. Burny, and P. Roy. 1999. Bovine leukemia virus Gag particle assembly in insect cells: formation of chimeric particles by domain-switched leukemia/lentivirus Gag polyprotein. Virology 265:308.

55. Luo, L., Y. Li, and C. Y. Kang. 1990. Expression of gag precursor protein and secretion of virus-like gag particles of HIV-2 from recombinant baculovirus-infected insect cells. Virology 179:874.

56. Morikawa, S., T. F. Booth, and D. H. Bishop. 1991. Analyses of the requirements for the synthesis of virus-like particles by feline immunodeficiency virus gag using baculovirus vectors. Virology 183:288.

57. Takahashi, R. H., K. Nagashima, T. Kurata, and H. Takahashi. 1999. Analysis of human lymphotropic T-cell virus type II-like particle production by recombinant baculovirus-infected insect cells. Virology 256:371.

58. Yamshchikov, G. V., G. D. Ritter, M. Vey, and R. W. Compans. 1995. Assembly of SIV virus-like particles containing envelope proteins using a baculovirus expression system. Virology 214:50.

59. Weldon, R. A., Jr., C. R. Erdie, M. G. Oliver, and J. W. Wills. 1990. Incorporation of chimeric gag protein into retroviral particles. J Virol 64:4169.

60. Andrawiss, M., Y. Takeuchi, L. Hewlett, and M. Collins. 2003. Murine leukemia virus particle assembly quantitated by fluorescence microscopy: role of Gag-Gag interactions and membrane association. J Virol 77:11651.

61. Leser, G. P., and R. A. Lamb. 2005. Influenza virus assembly and budding in raft-derived microdomains: a quantitative analysis of the surface distribution of HA, NA and M2 proteins. Virology 342:215.

62. Takeda, M., G. P. Leser, C. J. Russell, and R. A. Lamb. 2003. Influenza virus hemagglutinin concentrates in lipid raft microdomains for efficient viral fusion. Proc Natl Acad Sci U S A 100:14610.

63. Campbell, S. M., S. M. Crowe, and J. Mak. 2001. Lipid rafts and HIV-1: from viral entry to assembly of progeny virions. J Clin Virol 22:217.

64. Sandrin, V., and F. L. Cosset. 2006. Intracellular versus cell surface assembly of retroviral pseudotypes is determined by the cellular localization of the viral glycoprotein, its capacity to interact with Gag, and the expression of the Nef protein. J Biol Chem 281:528.

65. Salazar-Gonzalez, R. M., and S. J. McSorley. 2005. Salmonella flagellin, a microbial target of the innate and adaptive immune system. Immunol Lett 101:117.

66. Cuadros, C., F. J. Lopez-Hernandez, A. L. Dominguez, M. McClelland, and J. Lustgarten. 2004. Flagellin fusion proteins as adjuvants or vaccines induce specific immune responses. Infect Immun 72:2810.

67. Didierlaurent, A., I. Ferrero, L. A. Otten, B. Dubois, M. Reinhardt, H. Carlsen, R. Blomhoff, S. Akira, J. P. Kraehenbuhl, and J. C. Sirard. 2004. Flagellin promotes myeloid differentiation factor 88-dependent development of Th2-type response J Immunol 172:6922.

68. Tsujimoto, H., T. Uchida, P. A. Efron, P. O. Scumpia, A. Verma, T. Matsumoto, S. K. Tschoeke, R. F. Ungaro, S. Ono, S. Seki, M. J. Clare-Salzler, H. V. Baker, H. Mochizuki, R. Ramphal, and L. L. Moldawer. 2005. Flagellin enhances NK cell proliferation and activation directly and through dendritic cell-NK cell interactions. J Leukoc Biol 78:888.

69. Hayashi, F., T. K. Means, and A. D. Luster. 2003. Toll-like receptors stimulate human neutrophil function. Blood 102:2660.

70. Hayashi, F., K. D. Smith, A. Ozinsky, T. R. Hawn, E. C. Yi, D. R. Goodlett, J. K. Eng, S. Akira, D. M. Underhill, and A. Aderem. 2001. The innate immune response to bacterial flagellin is mediated by Toll-like receptor 5. Nature 410:1099.

71. Gewirtz, A. T., P. O. Simon, Jr., C. K. Schmitt, L. J. Taylor, C. H. Hagedorn, A. D. O'Brien, A. S. Neish, and J. L. Madara. 2001. Salmonella typhimurium translocates flagellin across intestinal epithelia, inducing a proinflammatory response. J Clin Invest 107:99.

72. Means, T. K., F. Hayashi, K. D. Smith, A. Aderem, and A. D. Luster. 2003. The Toll-like receptor 5 stimulus bacterial flagellin induces maturation and chemokine production in human dendritic cells. J Immunol 170:5165.

73. Smith, K. D., E. Andersen-Nissen, F. Hayashi, K. Strobe, M. A. Bergman, S. L. Barrett, B. T. Cookson, and A. Aderem. 2003. Toll-like receptor 5 recognizes a conserved site on flagellin required for protofilament formation and bacterial motility. Nat Immunol 4:1247.

74. Honko, A. N., N. Sriranganathan, C. J. Lees, and S. B. Mizel. 2006. Flagellin is an effective adjuvant for immunization against lethal respiratory challenge with Yersinia pestis. Infect Immun 74:1113.

75. Jeon, S. H., T. Ben-Yedidia, and R. Arnon. 2002. Intranasal immunization with synthetic recombinant vaccine containing multiple epitopes of influenza virus. Vaccine 20:2772.

76. Levi, R., and R. Arnon. 1996. Synthetic recombinant influenza vaccine induces efficient long-term immunity and cross-strain protection. Vaccine 14:85.

77. Lee, S. E., S. Y. Kim, B. C. Jeong, Y. R. Kim, S. J. Bae, O. S. Ahn, J. J. Lee, H. C. Song, J. M. Kim, H. E. Choy, S. S. Chung, M. N. Kweon, and J. H. Rhee. 2006. A bacterial flagellin, Vibrio vulnificus FlaB, has a strong mucosal adjuvant activity to induce protective immunity. Infect Immun 74:694.

78. Applequist, S. E., E. Rollman, M. D. Wareing, M. Liden, B. Rozell, J. Hinkula, and H. G. Ljunggren. 2005. Activation of innate immunity, inflammation, and potentiation of DNA vaccination through mammalian expression of the TLR5 agonist flagellin J Immunol 175:3882.

79. Ramos, H. C., M. Rumbo, and J. C. Sirard. 2004. Bacterial flagellins: mediators of pathogenicity and host immune responses in mucosa. Trends Microbiol 12:509.

80. Holsinger, L. J., and R. A. Lamb. 1991. Influenza virus M2 integral membrane protein is a homotetramer stabilized by formation of disulfide bonds. Virology 183:32.

81. Lamb, R. A., S. L. Zebedee, and C. D. Richardson. 1985. Influenza virus M2 protein is an integral membrane protein expressed on the infected-cell surface. Cell 40:627.

82. Holsinger, L. J., D. Nichani, L. H. Pinto, and R. A. Lamb. 1994. Influenza A virus M2 ion channel protein: a structure-function analysis. J Virol 68:1551.

83. Takeda, M., A. Pekosz, K. Shuck, L. H. Pinto, and R. A. Lamb. 2002. Influenza a virus M2 ion channel activity is essential for efficient replication in tissue culture. J Virol 76:1391.

84. Frace, A. M., A. I. Klimov, T. Rowe, R. A. Black, and J. M. Katz. 1999. Modified M2 proteins produce heterotypic immunity against influenza A virus. Vaccine 17:2237.

85. Neirynck, S., T. Deroo, X. Saelens, P. Vanlandschoot, W. M. Jou, and W. Fiers. 1999. A universal influenza A vaccine based on the extracellular domain of the M2 protein. Nat Med 5:1157.

86. Slepushkin, V. A., J. M. Katz, R. A. Black, W. C. Gamble, P. A. Rota, and N. J. Cox. 1995. Protection of mice against influenza A virus challenge by vaccination with baculovirus-expressed M2 protein. Vaccine 13:1399.

87. Treanor, J. J., E. L. Tierney, S. L. Zebedee, R. A. Lamb, and B. R. Murphy. 1990. Passively transferred monoclonal antibody to the M2 protein inhibits influenza A virus replication in mice. J Virol 64:1375.

88. De Filette, M., W. Min Jou, A. Birkett, K. Lyons, B. Schultz, A. Tonkyro, S. Resch, and W. Fiers. 2005. Universal influenza A vaccine: optimization of M2-based constructs. Virology 337:149.

89. De Filette, M., A. Ramne, A. Birkett, N. Lycke, B. Lowenadler, W. Min Jou, X. Saelens, and W. Fiers. 2006. The universal influenza vaccine M2e-HBc administered intranasally in combination with the adjuvant CTA1-DD provides complete protection. Vaccine 24:544.

90. Fiers, W., M. De Filette, A. Birkett, S. Neirynck, and W. Min Jou. 2004. A “universal” human influenza A vaccine. Virus Res 103:173.

91. Liu, W., Z. Peng, Z. Liu, Y. Lu, J. Ding, and Y. H. Chen. 2004. High epitope density in a single recombinant protein molecule of the extracellular domain of influenza A virus M2 protein significantly enhances protective immunity. Vaccine 23:366.

92. Fan, J., X. Liang, M. S. Horton, H. C. Perry, M. P. Citron, G. J. Heidecker, T. M. Fu, J. Joyce, C. T. Przysiecki, P. M. Keller, V. M. Garsky, R. Ionescu, Y. Rippeon, L. Shi, M. A. Chastain, J. H. Condra, M. E. Davies, J. Liao, E. A. Emini, and J. W. Shiver. 2004. Preclinical study of influenza virus A M2 peptide conjugate vaccines in mice, ferrets, and rhesus monkeys. Vaccine 22:2993.

93. Ionescu, R. M., C. T. Przysiecki, X. Liang, V. M. Garsky, J. Fan, B. Wang, R. Troutman, Y. Rippeon, E. Flanagan, J. Shiver, and L. Shi. 2006. Pharmaceutical and immunological evaluation of human papillomavirus viruslike particle as an antigen carrier. J Pharm Sci 95:70.

94. Hatziioannou, T., E. Delahaye, F. Martin, S. J. Russell, and F. L. Cosset. 1999. Retroviral display of functional binding domains fused to the amino terminus of influenza hemagglutinin. Hum Gene Ther 10:1533.

95. Li, Z. N., S. N. Mueller, L. Ye, Z. Bu, C. Yang, R. Ahmed, and D. A. Steinhauer. 2005. Chimeric influenza virus hemagglutinin proteins containing large domains of the Bacillus anthracis protective antigen: protein characterization, incorporation into infectious influenza viruses, and antigenicity. J Virol 79:10003.

96. Haynes, J. R., S. X. Cao, B. Rovinski, C. Sia, O. James, G. A. Dekaban, and M. H. Klein. 1991. Production of immunogenic HIV-1 viruslike particles in stably engineered monkey cell lines. AIDS Res Hum Retroviruses 7:17.

97. Rovinski, B., J. R. Haynes, S. X. Cao, O. James, C. Sia, S. Zolla-Pazner, T. J. Matthews, and M. H. Klein. 1992. Expression and characterization of genetically engineered human immunodeficiency virus-like particles containing modified envelope glycoproteins: implications for development of a cross-protective AIDS vaccine. J Virol 66:4003.

98. Fynan, E. F., R. G. Webster, D. H. Fuller, J. R. Haynes, J. C. Santoro, and H. L. Robinson. 1995. DNA vaccines: a novel approach to immunization. Int J Immunopharmacol 17:79.

99. Fynan, E. F., R. G. Webster, D. H. Fuller, J. R. Haynes, J. C. Santoro, and H. L. Robinson. 1993. DNA vaccines: protective immunizations by parenteral, mucosal, and gene-gun inoculations. Proc Natl Acad Sci U S A 90:11478.

100. Kodihalli, S., J. R. Haynes, H. L. Robinson, and R. G. Webster. 1997. Cross-protection among lethal H5N2 influenza viruses induced by DNA vaccine to the hemagglutinin. J Virol 71:3391.

101. Robinson, H. L., S. Lu, D. M. Feltquate, C. T. Tones, J. Richmond, C. M. Boyle, M. J. Morin, J. C. Santoro, R. G. Webster, D. Montefiori, Y. Yasutomi, N. L. Letvin, K. Manson, M. Wyand, and J. R. Haynes. 1996. DNA vaccines. AIDS Res Hum Retroviruses 12:455.

102. Drape, R. J., M. D. Macklin, L. J. Barr, S. Jones, J. R. Haynes, and H. J. Dean. 2006. Epidermal DNA vaccine for influenza is immunogenic in humans. Vaccine in press.

103. Kretzchmar, E., R. Geyer, and H. D. Klenk. 1994. Baculovirus infection does not alter N-glycosylation in Spodoptera frugiperda cells. Biol Chem Hoppe Seyler 375:23.

104. Lu, D., and A. J. Hickey. 2005. Liposomal dry powders as aerosols for pulmonary delivery of proteins. AAPS PharmSciTech 6:E641.

105. Cowdery, S., M. Frey, S. Orlowski, and A. Gray. 1976. Stability characteristics of freeze-dried human live virus vaccines. Dev Biol Stand 36:297.

106. Peetermans, J., G. Colinet, A. Bouillet, E. D'Hondt, and J. Stephenne. 1976. Stability of live, freeze-dried virus vaccines. Dev Biol Stand 36:291.

107. Yannarell, D. A., K. M. Goldberg, and R. N. Hjorth. 2002. Stabilizing cold-adapted influenza virus vaccine under various storage conditions. J Virol Methods 102:15.

108. Sampson, H. A., J. Bernhisel-Broadbent, E. Yang, and S. M. Scanlon. 1991. Safety of casein hydrolysate formula in children with cow milk allergy. J Pediatr 118:520.

109. Gambaryan, A. A. Tuzikov, G. Pazynina, N. Bovin, A. Balish, and A. Klimov. 2005. Evolution of the receptor binding phenotype of influenza A (H5) viruses. Virology (electronic publication ahead of print version).

110. Suzuki, Y, 2005. Sialobiology of Influenza: Molecular Mechanism of Host Range Variation of Influenza Viruses. Biological and Pharmaceutical Bulletin 28:399-408.

111. Latham, T., and J. M. Galarza. 2001. Formation of wild-type and chimeric influenza virus-like particles following simultaneous expression of only four structural proteins. J Virol. 75(13):6154-65.

112. Galarza, J. M., T. Latham, and A. Cupo. 2005. Virus-like particle (VLP) vaccine conferred complete protection against a lethal influenza virus challenge. Viral Immunol. 18(1):244-51.

1. A preparation of respiratory syncytial virus F polypeptide virus-like particles comprising a respiratory syncytial virus F polypeptide, wherein the virus-like particles do not comprise an enveloped virus core.

2. A preparation of respiratory syncytial virus F polypeptide virus-like particles comprising a respiratory syncytial virus F polypeptide, wherein the virus-like particles are pleomorphic, of non-uniform size, or of non-uniform shape.

3. A preparation of respiratory syncytial virus F polypeptide virus-like particles comprising a respiratory syncytial virus F polypeptide, wherein the virus-like particles do not comprise an enveloped virus core forming polypeptide.

4. The preparation of claim 1, wherein the virus-like particles comprise mammalian glycosylation.

5. The preparation of claim 1, wherein the virus-like particles are substantially non-aggregated with other virus-like particles.

6. The preparation of claim 1, wherein the virus-like particles are substantially not associated with viral vector particles.

7. The preparation of claim 1, further comprising an adjuvant in admixture with the virus-like particles.

8. The preparation of claim 7, wherein the adjuvant is located outside the virus-like particle.

9. The preparation of claim 7, wherein the adjuvant is located inside the virus-like particle.

10. The preparation of claim 7, wherein the adjuvant is covalently linked to the respiratory syncytial virus F polypeptide to form a covalent linkage.

11. The preparation of claim 1, wherein a neutralizing anti-RSV-F antibody binds to the respiratory syncytial virus F polypeptide.

12. The preparation of claim 11, wherein the neutralizing anti-RSV-F antibody is 9C5.

13. A method for producing a population of respiratory syncytial virus F polypeptide virus-like particles, comprising:
(a) providing an expression vector which expresses a respiratory syncytial virus F polypeptide;
(b) introducing the expression vector into a mammalian cell in a media; and
(c) expressing the respiratory syncytial virus F polypeptide to produce the respiratory syncytial virus F polypeptide virus-like particles, wherein the virus-like particles do not comprise an enveloped virus core.

14. A method for producing a population of respiratory syncytial virus F polypeptide virus-like particles, comprising:
(a) providing an expression vector which expresses a respiratory syncytial virus F polypeptide;
(b) introducing the expression vector into a mammalian cell in a media; and
(c) expressing the respiratory syncytial virus F polypeptide to produce the respiratory syncytial virus F polypeptide virus-like particles, wherein the virus-like particles are pleomorphic, of non-uniform size, or of nonuniform shape.

15. A method for producing a population of respiratory syncytial virus F polypeptide virus-like particles, comprising:
(a) providing an expression vector which expresses a respiratory syncytial virus F polypeptide;
(b) introducing the expression vector into a mammalian cell in a media; and
(c) expressing the respiratory syncytial virus F polypeptide to produce the respiratory syncytial virus F polypeptide virus-like particles, wherein the virus-like particles do not comprise an enveloped virus core forming polypeptide.

16. The method of claim 13, further comprising the step of recovering the respiratory syncytial virus F polypeptide virus-like particles from the media in which the mammalian cell is cultured.

17. The method of claim 13, wherein the expression vector is a viral vector.

18. The method of claim 17, wherein the viral vector is selected from the group consisting of: an adenovirus, a herpesvirus, a poxvirus and a retrovirus.

19. The method of claim 13, wherein the mammalian cell is selected from the group consisting of a BHK cell, a VERO cell, an HT1080 cell, an MRC-5 cell, a WI 38 cell, an MDCK cell, an MDBK cell, a 293 cell, a 293T cell, an RD cell, a COS-7 cell, a CHO cell, a Jurkat cell, a HUT cell, a SUPT cell, a C8166 cell, a MOLT4/clone8 cell, an MT-2 cell, an MT-4 cell, an H9 cell, a PM1 cell, a CEM cell, a myeloma cell, SB20 cell, a LtK cell, a HeLa cell, a WI-38 cell, an L2 cell, a CMT-93, and a CEMX 174 cell.

20. The method of claim 13, wherein a neutralizing anti-RSV-F antibody binds to the expressed respiratory syncytial virus F polypeptide.

21. The method of claim 20, wherein the neutralizing anti-RSV-F antibody is 9C5.

22. A method for treating or preventing respiratory syncytial virus infection comprising administering to a subject an immunogenic amount of the preparation of claim 1.

23. The method of claim 22, wherein the administering induces a protective immunization response in the subject.

24. The method of claim 22, wherein the administering is selected from the group consisting of subcutaneous delivery, transcutaneous delivery, intradermal delivery, subdermal delivery, intramuscular delivery, peroral delivery, oral delivery, intranasal delivery, buccal delivery, sublingual delivery, intraperitoneal delivery, intravaginal delivery, anal delivery and intracranial delivery.

25. A pharmaceutical composition comprising an immunogenic amount of the preparation of claim 1.

26. The pharmaceutical composition of claim 25 further comprising a pharmaceutically acceptable carrier.

27. A method for providing protection against respiratory syncytial virus infection comprising administering to a subject an immunogenic amount of the preparation of claim 1.

28. The method of claim 27, wherein the administering is selected from the group consisting of subcutaneous delivery, transcutaneous delivery, intradermal delivery, subdermal delivery, intramuscular delivery, peroral delivery, oral delivery, intranasal delivery, buccal delivery, sublingual delivery, intraperitoneal delivery, intravaginal delivery, anal delivery and intracranial delivery.


SEQUENCE LISTING (15 sequences)

SEQ ID NO: 1 - DNA, 20 bp, Artificial Sequence
  Synthesized Construct
tccatgacgttcctgacgtt

SEQ ID NO: 2 - DNA, 18 bp, Artificial Sequence
  Synthesized Construct
tctcccagcgtgcgccat

SEQ ID NO: 3 - DNA, 30 bp, Artificial Sequence
  Synthesized Construct
accgatgacgtcgccggtgacggcaccacg

SEQ ID NO: 4 - DNA, 24 bp, Artificial Sequence
  Synthesized Construct
tcgtcgttttgtcgttttgtcgtt

SEQ ID NO: 5 - DNA, 20 bp, Artificial Sequence
  Synthesized Construct
tccatgacgttcctgatgct

SEQ ID NO: 6 - DNA, 1650 bp, Artificial Sequence
  Synthesized Construct
atggaactgctgatcctgaaggctaacgctatcaccaccatcctgaccgctgtcaccttctgcttcgcctccggccagaacatcaccgaggaattctaccagtccacctgctccgctgtctccaagggttacctgtccgctctgcgcaccggctggtacacctccgtcatcaccatcgagctgtccaacatcaaggaaaacaagtgcaacggcaccgacgctaaggtcaagctgatcaagcaggaactggacaagtacaagaacgctgtcaccgagctgcagctgctgatgcagtccacccccgctaccaacaaccgcgctcgccgtgagctgccccgcttcatgaactacaccctgaacaacgccaagaaaaccaacgtcaccctgtccaagaagcgcaagcgccgcttcctgggtttcctgctgggtgtcggttccgctatcgcttccggtgtcgctgtctctaaggtcctgcacctggaaggcgaggtcaacaagatcaagtccgccctgctgtccaccaacaaggctgtcgtgtccctgtccaacggtgtctccgtcctgacctccaaggtgctggacctgaagaactacatcgacaagcagctgctgcccatcgtcaacaagcagtcctgctccatctccaacatcgagactgtcatcgagttccagcagaagaacaaccgcctgctggaaatcacccgcgagttctccgtcaacgctggtgtcaccacccctgtctccacctacatgctgaccaactccgagctgctgtccctgatcaacgacatgcccatcaccaacgaccaaaagaaactgatgtccaacaacgtccagatcgtccgccagcagtcctactctatcatgagcatcatcaaggaagaggtcctggcttacgtcgtccagctgcccctgtacggtgtcatcgacaccccctgctggaagctgcacacctcccccctgtgcaccaccaacaccaaggaaggttccaacatctgcctgacccgcaccgaccgcggctggttctgcgacaacgctggctctgtctccttcttcccccaagctgagacttgcaaggtccagtccaaccgcgtgttctgcgacaccatgaactccctgaccctgccctccgaggtcaacctgtgcaacgtcgacatcttcaaccccaagtacgactgcaagatcatgacctctaagaccgacgtgtcctcctctgtcatcacctccctgggtgctatcgtgtcctgctacggcaagaccaagtgcaccgcttccaacaagaaccgcggtatcatcaagaccttctccaacggttgcgactacgtgtccaacaagggcgtcgacaccgtgtccgtcggcaacaccctgtactacgtgaacaagcaggaaggcaagtccctgtacgtcaagggcgagcccatcatcaacttctacgaccccctggtgttcccctccgacgagttcgacgcttccatcagccaggtcaacgagaagatcaaccagtccctggctttcatccgcaagtccgacgagctgctgcacaacgtgaacgctggcaagtctaccaccaacatcatgatcaccactatcatcatcgtgatcatcgtcatcctgctgtctctgatcgctgtcggtctgctgctgtactaa

SEQ ID NO: 7 - protein, 549 aa, Artificial Sequence
  Synthesized Construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWFCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLY

SEQ ID NO: 8 - DNA, 1689 bp, Artificial Sequence
  Synthesized Construct
atggaactgctgatcctgaaggctaacgctatcaccaccatcctgaccgctgtgaccttctgcttcgcttccggccagaacatcaccgaggaattctaccagtccacctgctccgctgtgtccaagggttacctgtccgctctgcgtaccggttggtacacctccgtgatcaccatcgagctgtccaacatcaaagagaacaagtgcaacggcaccgacgctaaggtcaagctgatcaagcaggaactggacaagtacaagaacgctgtgaccgagctgcagctgctgatgcagtccacccccgctaccaacaaccgtgctcgtcgtgagctgccccgtttcatgaactacaccctgaacaacgccaagaaaaccaacgtcaccctgtccaagaagcgtaagcgtcgtttcctgggtttcctgctgggtgtgggtagcgctatcgcctccggtgtcgctgtctccaaggtgctgcacctcgagggcgaggtgaacaagatcaagtccgccctgctgtccaccaacaaggctgtggtgtccctgtccaacggtgtctccgttctgaccagcaaggtcttggacctgaagaactacatcgacaagcagctgctgcccatcgtgaacaagcagtcctgctccatctccaacatcgagactgtgatcgagttccagcagaagaacaaccgtctgctcgagatcacccgtgagttctccgtgaacgctggtgtcaccacccccgtgtccacctacatgctgaccaactccgagctgctgtccctgatcaacgacatgcccatcaccaacgaccagaaaaagctgatgtccaacaacgtgcagatcgtgcgtcagcagtcctactctatcatgagcatcatcaaagaggaagtcctggcttacgtggtgcagctgcccctgtacggtgtcatcgacaccccctgctggaagctgcacacctcccccctgtgcaccaccaacaccaaagagggttccaacatctgcctgacccgtaccgatcgtggttggttctgtgacaacgctggttccgtgtccttcttcccccaagctgagacttgcaaggtgcagtccaaccgtgtgttctgcgacaccatgaactccctgaccctgccctccgaggtgaacctgtgcaacgtggacatcttcaaccccaagtacgactgcaagatcatgacctctaagaccgacgtgtcctcctccgtcatcacctccctgggtgctatcgtgtcctgctacggcaagaccaagtgcaccgcttccaacaagaaccgcggtatcatcaagaccttctccaacggttgcgactacgtgtccaacaagggtgtcgataccgtgtccgtcggtaacaccctgtactacgtcaacaagcaggaaggcaagtctctgtacgtgaagggcgagcccatcatcaacttctacgaccccctggtgttcccctccgacgagttcgacgcttccatcagccaggtcaacgagaagatcaaccagtccctggctttcatccgtaagtccgacgagctgctgcacaacgtcaacgctggcaagtccaccaccaacatcctgtccatctactccaccgtggcttcctccctggctctggctatcatgatggctggtctgtccctgtggatgtgctccaacggctccctgcagtgccgtatctgcatctaataa

SEQ ID NO: 9 - protein, 561 aa, Artificial Sequence
  Synthesized Construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWFCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNILSIYSTVASSLALAIMMAGLSLWMCSNGSLQCRICI

SEQ ID NO: 10 - DNA, 1701 bp, Artificial Sequence
  Synthesized Construct
atggaactgctgatcctgaaggctaacgctatcaccaccatcctgaccgctgtgaccttctgcttcgcttccggccagaacatcaccgaggaattctaccagtccacctgctccgctgtgtccaagggttacctgtccgctctgcgtaccggttggtacacctccgtgatcaccatcgagctgtccaacatcaaagagaacaagtgcaacggcaccgacgctaaggtcaagctgatcaagcaggaactggacaagtacaagaacgctgtgaccgagctgcagctgctgatgcagtccacccccgctaccaacaaccgtgctcgtcgtgagctgccccgtttcatgaactacaccctgaacaacgccaagaaaaccaacgtcaccctgtccaagaagcgtaagcgtcgtttcctgggtttcctgctgggtgtgggtagcgctatcgcctccggtgtcgctgtctccaaggtgctgcacctcgagggcgaggtgaacaagatcaagtccgccctgctgtccaccaacaaggctgtggtgtccctgtccaacggtgtctccgttctgaccagcaaggtcttggacctgaagaactacatcgacaagcagctgctgcccatcgtgaacaagcagtcctgctccatctccaacatcgagactgtgatcgagttccagcagaagaacaaccgtctgctcgagatcacccgtgagttctccgtgaacgctggtgtcaccacccccgtgtccacctacatgctgaccaactccgagctgctgtccctgatcaacgacatgcccatcaccaacgaccagaaaaagctgatgtccaacaacgtgcagatcgtgcgtcagcagtcctactctatcatgagcatcatcaaagaggaagtcctggcttacgtggtgcagctgcccctgtacggtgtcatcgacaccccctgctggaagctgcacacctcccccctgtgcaccaccaacaccaaagagggttccaacatctgcctgacccgtaccgatcgtggttggttctgtgacaacgctggttccgtgtccttcttcccccaagctgagacttgcaaggtgcagtccaaccgtgtgttctgcgacaccatgaactccctgaccctgccctccgaggtgaacctgtgcaacgtggacatcttcaaccccaagtacgactgcaagatcatgacctctaagaccgacgtgtcctcctccgtcatcacctccctgggtgctatcgtgtcctgctacggcaagaccaagtgcaccgcttccaacaagaaccgcggtatcatcaagaccttctccaacggttgcgactacgtgtccaacaagggtgtcgataccgtgtccgtcggtaacaccctgtactacgtcaacaagcaggaaggcaagtctctgtacgtgaagggcgagcccatcatcaacttctacgaccccctggtgttcccctccgacgagttcgacgcttccatcagccaggtcaacgagaagatcaaccagtccctggctttcatccgtaagtccgacgagctgctgcacaacgtcaacgctggcaagtccaccaccaacggcacctaccagatcctgtccatctactccaccgtggcttcctccctggctctggctatcatgatggctggtctgtccctgtggatgtgctccaacggctccctgcagtgccgtatctgcatctaataa

SEQ ID NO: 11 - protein, 565 aa, Artificial Sequence
  Synthesized Construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWFCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNGTYQILSIYSTVASSLALAIMMAGLSLWMCSNGSLQCRICI

SEQ ID NO: 12 - DNA, 1620 bp, Artificial Sequence
  Synthesized Construct
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagtag

SEQ ID NO: 13 - protein, 538 aa, Artificial Sequence
  Synthesized Construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 14 - DNA, 1650 bp, Artificial Sequence
  Synthesized Construct
atggagctgctgatcctgaaggccaacgccatcaccaccatcctgaccgccgtgaccttctgcttcgcctccggccagaacatcaccgaggagttctaccagtccacctgctccgccgtgtccaagggctacctgtccgccctgcggaccggctggtacacctccgtgatcaccatcgagctgtccaacatcaaagaaaacaagtgcaacggcaccgacgccaaggtgaagctgatcaagcaggagctggacaagtacaagaacgccgtgaccgagctgcagctgctgatgcagtccacccctgccaccaacaaccgggccaggcgggagctgcctcggttcatgaactacaccctgaacaacgccaagaaaaccaacgtcaccctgtccaagaagcggaagcggcggttcctgggcttcctgctgggcgtgggctccgctatcgcctctggcgtggccgtgtctaaggtgctgcacctggagggcgaggtgaacaagatcaagtctgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagacagtgatcgagttccagcagaagaacaaccggctgctggaaatcacaagagagttctccgtcaacgctggtgtgaccactcctgtctctacttatatgctgaccaactccgagctgctgtccctgatcaacgacatgcctatcaccaacgaccagaaaaagctgatgtccaacaacgtgcagatcgtgcggcagcagtcctactctatcatgagcatcatcaaggaggaggtcctggcctacgtggtgcagctgcctctgtacggcgtgatcgacaccccttgctggaagctgcacacctcccccctgtgcaccaccaacaccaaggagggctccaacatctgcctgacccggaccgaccggggctggttctgcgacaacgccggctccgtgtccttctttccacaggccgagacatgcaaggtgcagtccaaccgggtgttctgcgataccatgaactccctgaccctgccttccgaggtgaacctgtgcaacgtggacatcttcaaccctaagtacgactgcaagatcatgacctctaagaccgacgtgtcctcctctgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagtctctgtacgtgaagggcgagcctatcatcaacttctacgaccctctggtgttcccttccgacgagttcgacgcctccatcagccaggtgaacgagaagatcaaccagtccctggccttcatccggaagtccgacgagctgctgcacaacgtgaacgctggcaagtctaccaccaaccccgaccactccgccgccaccaagccctccctgttcctgttcctggtgtccctgctgcacatcttcttcaagtgataa

SEQ ID NO: 15 - protein, 548 aa, Artificial Sequence
  Synthesized Construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWFCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNPDHSAATKPSLFLFLVSLLHIFFK